(19) United States
(12) Patent Application Publication
(10) Pub. No.: US 2016/0038570 A1
(43) Pub. Date: Feb. 11, 2016

(54) PREDICTING AND REDUCING ALLOIMMUNOGENICITY OF PROTEIN THERAPEUTICS

(71) Applicant: HAPLOMICS, INC., Atlanta, GA (US)

(72) Inventor: Tommy E. HOWARD, MANHATTAN BEACH, CA (US)

(21) Appl. No.: 14/868,327

(22) Filed: Sep. 28, 2015

Related U.S. Application Data
(63) Continuation of application No. 13/007,403, filed on Jan. 14, 2011, now abandoned.
(60) Provisional application No. 61/295,083, filed on Jan. 14, 2010.

(51) Int. Cl. A61K 38/37; G06F 19/18; G01N 33/68; C12Q 1/68

(52) U.S. Cl. CPC A61K 38/37; C12Q 1/6883; G06F 19/18; G01N 33/6893; C12Q 2600/172; C12Q 2600/156; C12Q 2600/106; G01N 2333/755; G01N 2800/52

(57) ABSTRACT

Methods of predicting the immunogenicity of a therapeutic protein in a subject are provided and the use of this method in selecting a protein for replacement therapy having the fewest immunogenic epitopes. The method is demonstrated by reference to ADAMTS13. Isolated allelic variants of ADAMTS13 that contribute to the variability in risk for both arterial and venous thrombotic disease development are provided. The allelic variants are identified as single nucleotide polymorphisms (ns-SNPs) in the ADAMTS13 gene, which result in haplotypes identified as H1 to H14. A method for improving outcomes of transfusions/transplant products is also provided by selection of haplotype matched therapeutics.

### CROSS-REFERENCE TO RELATED APPLICATIONS

This application is a continuation of U.S. patent application Ser. No. 13/007,403, filed on Jan. 14, 2011, which, in turn, claims benefit of U.S. Provisional Application No. 61/295,083, filed Jan. 14, 2010, each of which is hereby incorporated herein by reference in its entirety.

### FIELD OF THE INVENTION

The invention is generally in the field of diagnostic and therapeutics for detecting and/or predicting alloimmunogenic reactions following transfusion or transplantation.

### BACKGROUND OF THE INVENTION

The immunogenicity of protein-engineered therapeutics is of concern during the development and licensure of biologics (De Groot A S, et al. Clin Immunol 131:189-201 (2009)). Adding complexity to the issue, “biosimilars”, the equivalent of generics for biologics, appear to have a pathway for approval in the US Congress' recent health-care legislation (Walsh G. Nat Biotechnol 28:917-24 (2010)). Interchangeability is central for the economic promise of a biosimilar product to be realized but the potential for immunogenicity will likely prevent products from being freely substitutable.

Recent studies have demonstrated that T-cell epitopes play an essential role in eliciting anti-drug antibodies (ADAs) against therapeutic proteins (Barbosa M D, et al. Clin Immunol 118:42-50 (2006)). Considerable progress has been made in the assessment of T-cell epitopes using computational, in vitro and ex vivo methods (De Groot A S, et al. Curr Opin Pharmacol 2008; 8:620-6). Unfortunately, this progress has not translated into accurate predictions of immunogenicity. Not that all patients develop inhibitory antibodies. However, some individuals, racial and/or ethnic groups, or other sub-populations have a stronger immunogenic reaction than others. Current strategies to predict immunogenicity focus largely on identifying epitopes during pre-clinical development based on the postulate that engineering such epitopes will result in a protein that is universally less immunogenic within the entire population (De Groot A S, et al. Clin Immunol 131:189-201 (2009)). Such strategies are likely to be insufficient due to the substantial genomic variability within the patient population. Thus, an alternative decision tree is needed that takes a personalized approach to predicting (and eventually circumventing) immunogenicity. For example, computer-based computational methods and algorithms are needed that accurately predict immunogenicity. Such prediction algorithms would be invaluable during the preclinical stage of drug development as well as in identifying and stratifying each individual's risk of developing inhibitory ADAs.

Sickle cell disease (SCD) is an inherited disorder due to homozygosity for the abnormal hemoglobin, hemoglobin S (HbS). This abnormal hemoglobin S is caused by the substitution of a single base in the gene encoding the human B-globin subunit. Its reach is worldwide, affecting predominantly people of equatorial African descent, although it is found in persons of Mediterranean, Indian, and Middle Eastern lineage. SCD is considered a pre-thrombotic state, since certain characteristics of sickle cells such as abnormal adhesivity and absence of membrane phospholipid asymmetry are involved in the thrombotic process (Marfaing-Koka, et al., Nouv Rev Fr Hamatol, 35:425-430 (1993)). Most of the morbidity of SCD appears to be related to the appearance of occlusion of the microvasculature, resulting in widespread ischemia and irreversible organ damage. Vaso-occlusion results in recurrent painful episodes (sometimes called sickle cell crisis) and a variety of serious organ system complications among which infection, acute chest syndrome, stroke, splenic sequestration are among the most debilitating. Vaso-occlusion accounts for 90% of hospitalizations in children with SCD, and can lead to life-long disabilities and/or early death.

The pathophysiology of vaso-occlusion is complex and involves polymerization of deoxygenated hemoglobin S, which produces sickled cells that cause vaso-occlusion. Abnormal interactions between these poorly deformable sickled cells and the vascular endothelium result in dysregulation of vascular tone, activation of monocytes, upregulation of adhesion molecules and a shift toward a procoagulant state. Current thought suggests that vaso-occlusion is a two-step process. First, deoxygenated sickle cells expressing pro-adhesive molecules adhere to the endothelium to create a nidus of sickled cells, then sickled cells accumulate behind this blockage to create full blown vaso-occlusion.

Most patients with sickle cell disease can be expected to survive into adulthood, but still face a lifetime of crises and complications, including chronic hemolytic anemia, vaso-occlusive crises and pain, and the side effects of therapy. Currently, most common therapeutic interventions include blood transfusions, opioid and hydroxyurea therapies (Ballas, Cleveland Clin. J. Med., 66:48-58 (1999)). Blood transfusions are geared towards replacing the patient's red blood cells (RBCs) with transfused RBCs and hydration that thus decrease the percentage of sickled RBCs in the bloodstream. Although transfusion therapy is effective in reducing vaso-occlusive crises, patient response is highly variable, and transfusion therapy also carries the risk of alloimmunogenic reactions. There is currently a need to improve the efficacy of such therapies and reduce the likelihood of developing potentially fatal antibody-based inhibitors and either macro- or micro-vascular thrombotic diseases.

Multiple adhesion molecules have been shown to participate in SS-RBC/endothelium interactions. These include fibrinogen and fibronectin (Wautier, et al., J Lab Clin Med, 101:911-20 (1983); Kasschau, et al., Blood, 87:771-80 (1996)), laminin (Hillery, et al., Blood, 87:4879-861(1996); Lee, et al., Blood, 92:2951-8 (1998)) and thrombospondin (Sugihara, Blood, 80:2634-42 (1992); Hillery, et al., Blood, 94:302-91(999)) and von Willebrand factor (“vWF”; Wick, et al., 80:905-10 (1987); Kaul, et al., Blood 81:2429-3 (1993)).

ADAMTS13 is a plasma protease that decreases the adhesiveness of vWF by cleaving vWF. ADAMTS13 is an important hemostatic factor in modulating a number of thrombotic diseases, e.g. stroke and myocardial infarction. It is also believed that ADAMTS13 activity is a factor in the development of thrombotic thrombocytopenic purpura (TTP), a thrombotic microangiopathy characterized by hemolytic anemia, thrombocytopenia, and ischemic complications in the brain and other organs. The original gene sequence for ADAMTS13 including several loss-of-function mutations that contribute to deficiencies in ADAMTS13 activity and cause or increase the likelihood of developing thrombotic thrombocytopenic purpura (TTP) are disclosed in U.S. Pat. Nos. 7,517,522 and 7,037,658. U. S. Published application No. 20090317375 discloses the administration of recombinant ADAMTS13 to treat or prevent infarction, by increasing patients' ADAMTS13 activity. A common allele of ADAMTS13 produced by a consensus ADAMTS13 gene sequence and a short, specific amino acid sequence of ADAMTS13 have both been described and are in commercial development. However, there are no studies relating to multiple common wild-type ADAMTS13 allelic variants (and likely multiple mild loss-of-function variants) in human populations that may contribute to the large inter-individual variability in risks that have been observed for arterial and venous thrombotic disorders. Further, there has been no correlation of the multiple common (wild-type and likely mild loss-of-function type) ADAMTS13 allelic variants in human populations with the development of alloantibodies against individuals' two ADAMTS13 alleles (termed ‘self’) through exposure to other, non-self ADAMT13 alleles (termed ‘foreign’) and, in turn, the development of macrovascular and/or microvascular thrombotic diseases.

It is an object of the present invention to provide methods to predict immunogenicity of protein-engineered therapeutics.

It is a further object of the invention to provide methods of selecting the least immunogenic protein for replacement therapy in a subject.

It is also an object of the present invention to provide methods of treating hemophilia in a subject with an intron-22 inversion (1221) in the F8 gene.

It is also an object of the present invention to provide recombinant allelic variants of ADAMTS13 contributing to the variability in risk for both arterial and venous thrombotic disease development.

It is also an object of the present invention to provide a method for reducing incidences of alloimunogenic reactions following transfusions/transplant of ADAMTS13 containing products.

It is further an object of the present invention to provide screening methods for allelic variants of ADAMTS13 contributing to the variability in risk for both arterial and venous thrombotic disease development.

### SUMMARY OF THE INVENTION

Methods of predicting the immunogenicity of a therapeutic protein (e.g., for use in replacement therapy) in a subject are provided. These methods can involve identifying one or more epitopes in the therapeutic protein; identifying the MHC-II molecules present on the cells in the subject; and determining the binding affinity of each epitope to the MHC-II molecules on cells in the subject. The presence of an epitope that binds with high affinity to MHC-II molecules on the cells in the subject can be an indication that the therapeutic protein is immunogenic in the subject.

The one or more epitopes can be identified by determining sequence variation between the therapeutic protein and an endogenous protein in the subject, wherein an amino acid fragment comprising the sequence variation in the therapeutic protein is an epitope for the subject. The subject's endogenous protein sequence can be identified by determining the nucleic acid sequence of the gene encoding the endogenous protein in the subject. Alternatively, the subject's endogenous protein sequence can be identified by determining the effect of nucleic acid sequence on intracellular expression of the endogenous protein. Intracellular protein expression is determined, for example, by immunoassay or in silico.

The binding affinity of each epitope to MHC-II molecules on the subject's cells can also be determined in silico. Preferably, the MHC-II molecules present on the cells in the subject are identified by genotyping the subject's MHC-II haplotype. Alternatively, the MHC-II molecules present on the cells in the subject are identified by determining the MHC-II frequencies in the subject's racial or ethnic subpopulation. The concentration of the MHC-II molecules on the subject's cells can also be assessed. The presence of an epitope that binds with high affinity to MHC-II molecules that are expressed at high concentration on the cells in the subject is an indication that the infused protein is immunogenic in that subject.

Also provided is a method of selecting a protein for replacement therapy in a subject that involves predicting the immunogenicity of each candidate thereapeutic protein and selecting a candidate protein for use in replacement therapy in the subject having the fewest epitopes (preferably none) that bind with high affinity to the MHC-II molecules on cells in the subject.

A method of treating a subject in need of protein replacement therapy with a therapeutic protein is also provided. The method can involve identifying one or more epitopes in the therapeutic protein; identifying the MHC-II molecules present on the cells in the subject; determining the binding affinity of each epitope to the MHC-II molecules on cells in the subject; identifying one or more immunogenic epitopes in the thereapeutic protein that bind with high affinity to MHC-II molecules on the cells in the subject; and vaccinating the subject with one or more peptides including the one or more immunogenic epitopes. The one or more peptides can be administered to the subject with immunosuppressants.

Also provided is a method predicting the immunogenicity of FVIII protein in a subject with an intron-22 inversion (1221) in the F8 gene. The method can involve identifying the MHC-II molecules present on the cells in the subject and determining the binding affinity of a peptide comprising the amino acids encoded by the exon-22/exon-23 junction sequence in the F8 gene to the MHC-II molecules on cells in the subject. In this method, binding of the peptide with high affinity to the MHC-II molecules on the cells in the subject is an indication that FVIII protein is immunogenic in the subject.

A method of treating hemophilia in a subject with an intron-22 inversion (1221) in the F8 gene is also provided that involves predicting the immunogenicity of FVIII protein in the subject by the above method, and vaccinating the subject, preferably an infant, with a peptide containing an amino acid sequence encoded by the exon-22/exon-23 junction sequence in the F8 gene.

Isolated allelic variants of ADAMTS13 that contribute to the variability in risk for both arterial and venous thrombotic disease development have been identified as nonsynonymous single nucleotide polymorphisms (ns-SNPs) in the ADAMTS13 gene which result in different ADAMTS13 haplotypes (H). The ns-SNPs result in variations at positions 7, 448, 456, 458, 625, 740, 900, 982, 998 1033 and 1226 in the ADAMTS13 protein. The amino acid variations result in the following amino acids at positions 7, 448, 456, 458, 625, 740, 900, 982, 1033 and 1226: H1 (SEQ ID NO:1), H2 (SEQ ID NO:2); H3 (SEQ ID NO:3); H4 (SEQ ID NO:4); H5 (SEQ ID NO:5); H6 (SEQ ID NO:6); H7 (SEQ ID NO:7); H8 (SEQ ID NO:8); H9 (SEQ ID NO:9); H11 (SEQ ID NO:11); H12 (SEQ ID NO:12); H13 (SEQ ID NO:13); H14 (SEQ ID NO:14).

A method for improving outcomes of transfusions/transplant products is provided by identifying the ADAMTS13 haplotype of a transfusion/transplant replacement product, identifying the ADAMTS13 haplotype of the recipient and then administering a haplotype-matched transfusion product to the subject based on the results. In a preferred embodiment, the ADAMTS13 haplotype is H1, H2 H3, H4, H5, H6, H7, H8, H9, H11, H12, H13, or H14. In some embodiments the replacement product is blood or plasma. In other embodiments the replacement product is recombinant ADAMTS13.

Methods for screening for allelic variants of ADAMTS13 contributing to the variability in risk for both arterial and venous thrombotic disease development are provided. In one embodiment, the methods include obtaining a sample from a subject and identifying the SNPs C463T, C2105G, G2131T, C2133T, C2615G, G2637A, G2981A, C3462T, C3462T, G3707A, C3755G, G3860A, and C440T in the ADAMTS13 gene.

Also disclosed is a method of blood plasma pooling which includes the steps of detecting a haplotype in an ADAMTS13 gene of a blood plasma donor and placing blood plasma of the blood plasma donor in an appropriate pool based on the results. In some embodiments the method of pooling blood plasma includes the steps of detecting a haplotype in a ADAMTS13 gene of a whole blood donor, receiving whole blood from the whole blood donor, separating plasma from the whole blood, and pooling the plasma with plasma obtained from other donors with the same haplotype where possible or most closely matched haplotype. Pooled blood plasma products obtained through this method, in which the pooled plasma is homogenous or enriched in H1, H2, H3, H4, H5, H6, H7, H8, H9, H10, H11, H12, H13 or H14 are also provided.

### BRIEF DESCRIPTION OF THE DRAWINGS

FIG. 1 is an illustration of the ADAMTS13 gene showing its 29 exons (triangles), 28 introns (lines), and the exonic position of 11 ns-SNPs identified by SeattleSNPs® via resequencing in a group of 47 unrelated individuals.

FIG. 2A shows the domain-structure and variable positions encoded by ns-SNPs (whose minor alleles are to the right). FIG. 2B shows 14 structurally-distinct forms (designated here as haplotypes 1 through 14), which are encoded by the naturally-occurring allelic combinations of these 11 ns-SNPs. The frequency (F) characteristics of each haplotype in the variation discovery collection (N=47) studied by SeattleSNPs® is shown in the total (T) group, independent of race, and in either the 24 African-American (AA) or 23 Caucasian-American (CA) subjects alone.

FIG. 3A is a bar graph showing the levels of Factor VIII (FVIII) gene (F8)-derived mRNAs (fold change (2^{ΔΔCp}), which contain (at least) either exons 1 to 22, exons 23 to 26, or the exon 22-exon 23 junction of the F8 gene, detected using q-RT-PCR, and then normalized to the mRNA levels encoded by the housekeeping gene GAPDH, both in a normal individual (first, third, and fifth bars) and in a patient with severe Hemophilia A (HA) with the intron-22 (I22)-inversion (I22I) (second, fourth, and sixth bars). (Mean±SD, n=3). FIGS. 3B-3E are flow cytometry histograms showing the results of the experimental attempts to detect the presence of the FVIII protein (full-length or fragments) either intracellularly or within the cell (plasma) membrane using anti-human-FVIII antibodies (unfilled histograms for ESH5, Ab41188, and ESH8)—and isotype control antibodies (filled histograms for IgG2a and IgG1) as negative controls—in permeabilized (FIGS. 3C and 3E) and non-permeabilized (FIGS. 3B and 3D) cells, respectively, obtained from a normal individual (FIGS. 3B and 3C) and HA patient with the 1221 (FIGS. 3D and 3E). Binding of antibodies to protein was detected using an Alexa Fluor 488 labeled goat anti-mouse IgG secondary antibody. Each histogram depicts the fluorescence intensities of 10,000 cells. FIGS. 3F-3H are graphs depicting the mean fluorescence from data in FIGS. 3B and 3E for ESH5 (FIG. 3F), ESH8 (FIG. 3G), and Ab41188 (FIG. 3H) compared to isotype controls in the normal individual (second and fourth bars) and the HA patient with the 1221 (first and third bars). FIGS. 3I and 3J are graphs showing flow cytometry counts using anti-FVIII antibodies (Ab41188 or ESH8) in permeabilized cells from the normal individual (FIG. 3I) and the HA patient with the 1221 (FIG. 3J) treated with increasing concentrations (0, 1, 2, or 5 μM) of the Smart Pool siRNA specific to the F8 mRNA. FIG. 3K is a graph showing the Smart Pool siRNA-mediated decrease in FVIII protein levels (median fluorescence) plotted as a function of siRNA concentration ([μM]).

FIG. 4A is a diagram depicting computational predictions of the binding of overlapping peptides in the FVIII protein (top axis) to MHC Class II alleles that occur most frequently in the human population (left axis). The region of the protein that is shown (amino acids 2095 to 2160) spans the exon22-exon23 junction and the sequence (SEQ ID NO:25) is at the top of the heat map. The binding affinity is shown as a percentile score as compared to 5 million random peptides from the Swiss Prot data base where a lower percentile score indicates tighter binding. The heat map has been generated using a scale of 0-5% (instead of 0-100%) to emphasize differences between different tight binding peptides. The large blank area on either side of the junction indicates that most peptides do not bind with high affinity to any of the HLA alleles depicted. The columns are in the region of the amino-acids Y2105 and 82150. HA patients with missense mutations at these positions frequently develop inhibitory antibodies. The circles adjacent to the MHC Class II alleles show the ethnic distribution of these alleles; the unfilled circles show those that occur most frequently in Caucasians, the black circles those that occur most frequently in individuals of African descent and the grey circles those that occur in both populations. FIG. 4B is a diagram depicting an immunogenicity score as a function of amino acid position in mature FVIII protein based on the number of HLA alleles that the peptides at each location bind to. The region of the protein that is shown (amino acids 2095 to 2160) spans the exon22-exon23 junction and the sequence (SEQ ID NO:25) is given at the top of the heat map. The diagram illustrates that there is a local minima in the region of the exon-22/exon-23 junction.

FIGS. 5A and 5B are diagrams depicting the structure of the wild-type F8 gene (FIG. 5A) and the 1221 (FIG. 5B).

FIG. 6 is a diagram depicting nonsynonymous-SNPs (ns-SNPs) and the FVIII proteins they encode, only two of which have the amino acid sequences found in recombinant FVIII molecules used clinically. These ns-SNPs encode the following amino acid substitutions, respectively: proline for glutamine at position 334 (Q334P), histidine for arginine at position 484 (R484H), glycine for arginine at position 776 (R776G), glutamic acid for aspartic acid at position 1241 (D1241E), lysine for arginine at position 1260 (R1260K), and valine for methionine at position 2238 (M2238V). The numbering systems used to designate the positions of the amino acid substitutions encoded are based on their residue locations in the mature circulating form of wild-type FVIII. R484H and M2238V are components of the A2- and C2-domain immunodominant epitopes that include residues arginine at position 484 to isoleucine at position 508 and glutamate at position 2181 to valine at position 2243, respectively. The inset shows the two full-length recombinant FVIII proteins used in replacement therapy, Kogenate (same as Helixate) and Recombinate (same as Advate). The B-domain deleted recombinant FVIII protein, Refacto (same as Xyntha), does not contain the ns-SNP site differentiating Kogenate and Recombinate (D1241E).

FIG. 7A is a diagram depicting the genomic structure of the wild-type F8 gene. F8 has 26 exons (exons 3-20, 24, and 25 are not shown), which are oriented centromerically, and is located approximately one Mb from the telomere on the long-arm of the X-chromosome. Intron-22 (122) is approximately 33 kb and contains an approximately 9.5 kb sequence (int22h-1), that includes F8A, a single exon gene oriented telomerically, and exon-1 of a five exon, centromerically-oriented gene, F8_{B}, that shares exons 2-5 (exons 3 and 4 not shown) with F8 (exons 23-26). Two sequences homologous to int22h-1 (int22h-2 and int22h-3) are located telomeric to F8. Int22h-2 and int22h-3 are each part of a larger approximately 50 kb duplication contributed primarily by a approximately 40 kb sequence shown by the two pink rectangles. FIG. 7B is a diagram depicting direct homologous recombination of int22h-1 with int22h-3. FIG. 7C is a diagram depicting structure of F8 gene following homologous recombination and intra-chromosomal rearrangement.

FIGS. 8A-B consist of a diagram depicting the genomic structure of wild-type (FIG. 8A) and I22-inverted F8 (FIG. 8B).

FIG. 9 shows amino acids 2105 and 2150 of FVIII's C1 domain and exon-22/exon-23 junction (SEQ ID N0:26). The arrows identify 1221 breakpoint between residues 2124 and 2125. Y2105 and R2150 (*) are sites of recurrent missense mutations strongly associated with inhibitors. The top row illustrates missense mutations that have been identified in patients that have not developed inhibitors.

FIG. 10 is a diagram depicting immunogenicity potential (%) of wild-type FVIII-derived peptides for nine HLA-DRBI proteins defined as the percent of the proteins that bind with high affinity as a function of amino acid position. The line labeled “all” designates the immunogenicity potential for those peptides that bind with high affinity to those DRB1 alleles found in both black African and white European populations. The line labeled “Africans” designates the immunogenicity potential for those peptides that bind with high affinity to the DRB1 alleles found only in black Africans while the line labeled “Caucasians” designates the immunogenicity potential of those peptides that bind with high affinity to the DRB1 alleles found only in white Europeans.

FIG. 11 illustrates individualized pharmacogenetic parameters for determining the immunogenicity of an infused protein.

FIG. 12A is a plot illustrating the predicted percentile ranks for overlapping peptides spanning the entire FVIII sequence to HLA-DRB1*1501. Only the peptides predicted to bind this MHC-II molecule are depicted. FIG. 12B is a graph showing true positive rate for immunogenicity score computed at each of the FVIII positions as a function of false positive rate, indicating that the immunogenicity score significantly discriminates between positive and negative positions (area under the ROC curve=0.66; Mann-Whitney U p-value 0.0086). FIG. 12C is a diagram depicting computational predictions of the binding of overlapping peptides in the FVIII protein (top axis) to MHC Class II alleles (left axis). FIG. 12D is a diagram depicting immunogenicity potential (%) of regions of FVIII with the three highly recurrent HA-causing missense mutations (Y2105C, R2150H, and W2229C) for HLA-DRB1 proteins defined as the percent of the proteins that bind with high affinity as a function of amino acid position. Peptides that incorporate Y2105 and 82150 show high affinity (low percentile binding rank) for most MHC-II molecules. Peptides that incorporate W2229 appear not to bind most MHC-II molecules, however, the heat map shows that these peptides do bind with very high affinity to the MHC-II molecule HLA-DRB1*0301.

### DETAILED DESCRIPTION OF THE INVENTION

Methods for predicting alloimmunogenic of a therapeutic protein, such as a protein for replacement therapy, have been identified. Multiple allelic variants of ADAMTS13 contributing to alloantibody (and occasionally to autoantibody) formation and the development of macrovascular and/or microvascular thrombotic diseases have also been identified.

### DEFINITIONS

The term “immunity,” “immunogenic,” and “antigenic” refer to the ability of a protein, such as a therapeutic protein for replacement therapy, to induce an immune reaction in a subject.

The term “alloimmunity” and “alloimmunogenic” refer to immunity in a subject to an antigen from another individual of the same species. An “alloantigen” is an antigen that is present in some members of the same species, but is not common to all members of that species. If an alloantigen is presented to a member of the same species that does not have the alloantigen, it will be recognized as foreign by the self-recognition system, e.g., Major Histocompatibility Complex (MHC) complex.

The term “tolerization” refers to the induction of tolerance of the immune system to a particular antigen, which would otherwise induce an immune response. Tolerized proteins, e.g., endogenous proteins, are considered as self by the immune system and do not induce an immune response.

The term “epitope”, typically an amino acid sequence of about three to seven amino acids, refers to a portion of an antigen that is recognized by the immune system as non-self. The term refers to protein fragments (including single amino acids) that are not present in a subject's endogenous protein and therefore can be recognized as non-self by the immune system.

The term “sequence variation” refers to any difference between two or more amino acids sequences or the nucleic acid sequences encoding the amino acid sequences.

A “single nucleotide polymorphism” (or SNP) refers to a genetic locus of a single base which may be occupied by one of at least two different nucleotides. Single nucleotides may be changed (substitution), removed (deletion) or added (insertion) to a polynucleotide sequence. Insertion and deletion SNPs may shift the translational frame. A nonsynonymous SNP includes changes in the nucleic acid code that lead to an altered or different polypeptide sequence. A nonsynonymous SNP may either be missense or nonsense, where a missense change results in a different amino acid, while a nonsense change results in a premature stop codon.

The term ““ADAMTS13”” refers to a disintegrin and metalloproteinase with a thrombospondin type 1 motif, member 13. ADAMTS13 has been identified as a unique member of the metalloproteinase gene family, ADAM (a disintegrin and metalloproteinase), whose members are membrane-anchored proteases with diverse functions. ADAMTS family members are distinguished from ADAMs by the presence of one or more thrombospondin 1-like (TSP1) domain(s) at the C-terminus and the absence of the EGF repeat, transmembrane domain and cytoplasmic tail typically observed in ADAM metalloproteinases. The ADAMTS13 protein is secreted in blood and degrades large vWf multimers, decreasing their activity.

“Isolated”” refers to material removed from its original environment (e.g., the natural environment if it is naturally occurring), and thus is altered ““by the hand of man”” from its natural state. For example, an isolated polynucleotide could be part of a vector or a composition of matter, or could be contained within a cell, and still be ““isolated”” because that vector, composition of matter, or particular cell is not the original environment of the polynucleotide. The term ““isolated”” does not refer to genomic or cDNA libraries, whole cell total or mRNA preparations, genomic DNA preparations (including those separated by electrophoresis and transferred onto blots), sheared whole cell genomic DNA preparations or other compositions where there are no distinguishing features of the polynucleotide/sequences.

The term “subject” refers to any individual who is the target of administration, typically a human.

The term “predict” refers to the ability of a method to prognose an outcome based on medical and diagnostic information. The term does not denote an absolute certainty. In some embodiments, the term refers to the ability to determine an outcome with a statistical certainty.

The term “treatment” refers to the medical management of a patient with the intent to cure, ameliorate, stabilize, or prevent one or more symptoms of disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. The term includes palliative treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder.

As used herein, a “sample” from a subject means a tissue, organ, cell, cell lysate, biomolecule derived from a cell or cellular material (e.g. a polypeptide or nucleic acid), or body fluid from a subject. Non-limiting examples of body fluids include blood, plasma, serum, cerebrospinal fluid, interstitial fluid, amniotic fluid, and semen.

### I. Compositions

A. ADAMTS13 Allelic Variants

Isolated nucleic acid and amino acid allelic variants of ADAMTS13 contributing to the variability in risk for both arterial and venous thrombotic disease development and more effective treatment through a similar mechanism involving either matched blood transfusions, matched replacement therapy, and/or matched cell and organ transplants have been identified. The allelic variants of ADAMTS13 are designated H1 to H14 and are variants of the ADAMTS13 gene provided by GenBank No. DQ422807.

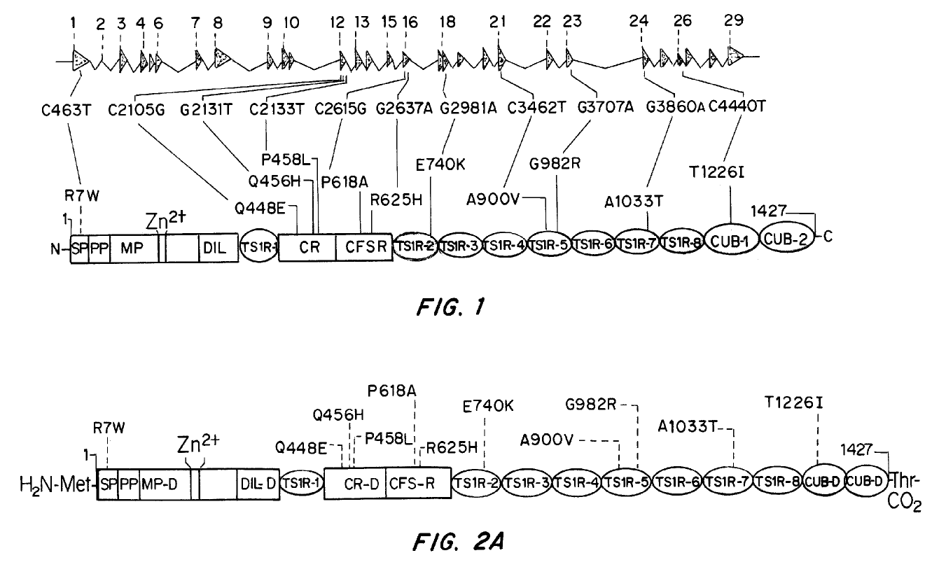

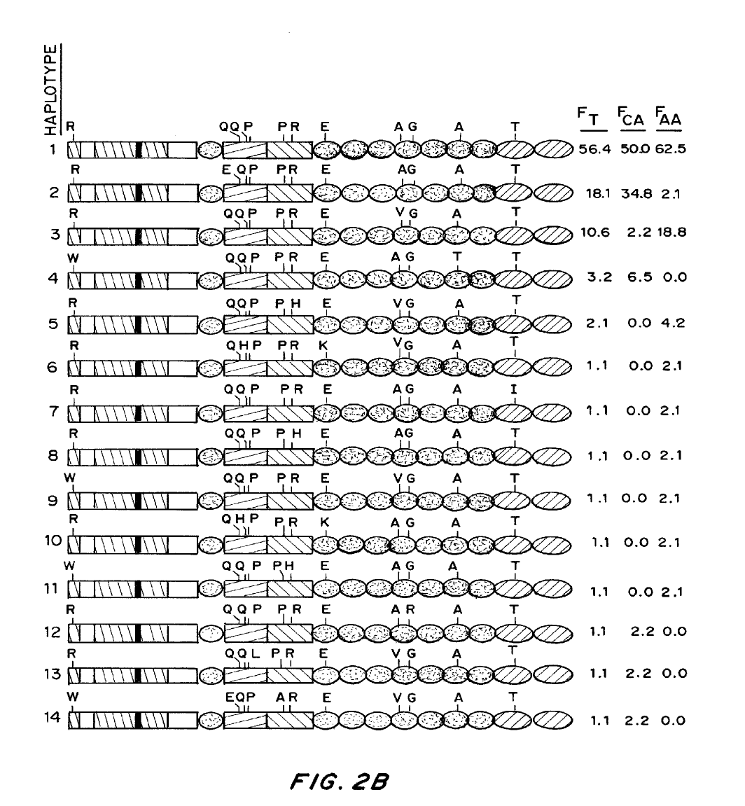

An ongoing resequencing-based, genome-wide variation study of 47 unrelated, healthy individuals (24 blacks and 23 whites) identified 11 ns-SNPs in ADAMTS13. By analyzing this genotype data, using the expectation-maximization algorithm in GENECOUNTING, alleles of these 11 ns-SNPs were found to exist in numerous combinations (“haplotypes”) that encode 14 structurally-distinct forms of ADAMTS13 (FIG. 2B). The minor allele (MA) of each ns-SNP is shown on the right of its nucleotide location using an mRNA-based numbering system with the transcription initiation site indicated as base 1. The 11 ns-SNPs are shown in FIG. 1 as C463T, C2105G, G2131T, C2133T, C2615G, G2637A, G2981A, C3462T, C3462T, G3707A, G3860A and C440T. The resultant amino acid allelic variations in the protein sequence are R7W, Q448E, Q456H, P458L, R625H, E740K, A900V, G982R, A1033T and T12261 (minor allele in bold). The MA of each variable residue encoded by a ns-SNP is shown on the right of its location in the protein using an amino acid numbering system based on the translation initiation site indicated as residue 1. The domain structure of ADAMTS13 is in FIGS. 1 and 2A, as are the positions of the variable protein sites encoded by the 11 biallelic ns-SNPs, which are located in the signal peptide (SP), three of the eight thrombospondin type-1 repeats (TS1R; 2, 5 and 7), both the cysteine-rich (CR) and cysteine-free spacer region (CFSR), and the first of two complement, uEGF, and bone morphogenesis (CUB) domains (FIG. 1A). No ns-SNPs were identified in either the propeptide (PP), metalloprotease (MP) domain, zinc-binding (Zn²) motif, or disintegran-like (DIL) domain in the relatively small variation discovery group scanned by SeattleSNPs. The minor allele frequency (MAF) in the overall variation discovery group, independent of ethnicity, and the predicted affect on ADAMTS13 activity based on POLYPHEN analysis is shown in Table 1:

TABLE 1
ns-SNP minor allele frequency (MAF) and Prediction

| ns-SNPs | MAF | Prediction |
|---|---|---|
| Arg0007Trp | 6.0% | Damaging |
| Gln0448Glu | 19.0% | Benign |
| Gln0456His | 2.0% | Benign |
| Pro0458Leu | 1.0% | Damaging |
| Pro0618Ala | 1.0% | Damaging |
| Arg0625His | 4.0% | Benign |
| Glu0740Lys | 2.0% | Benign |
| Ala0900Val | 17.0% | Benign |
| Gly0982Arg | 1.0% | Damaging |
| Ala1033Thr | 3.0% | Benign |
| Thr1226Ile | 1.0% | Benign |

The frequency (F) characteristics of each haplotype in the variation discovery collection (N=47) studied by SeattleSNPs is shown in the total (T) group, independent of race, and in either the 24 African-American (AA) or 23 Caucasian-American (CA) subjects alone (FIG. 2B). ADAMTS13 in 191 individuals who were either donors or recipients of kidney transplants was also resequenced and the existence of these 11 ns-SNPs and 14 ADAMTS13 haplotypes were confirmed. An additional ns-SNP was also identified. Previously, in a group of about 200 unrelated predominantly white American subjects (which also contained some black, Hispanic, and Asian individuals), the existence and frequency of all alleles of the 11 ns-SNPs was confirmed. A new ns-SNP, C3755G (which encodes Leu998Val) was also identified in one black subject whose less frequent minor allele defined a new black-restricted ADAMTS13 haplotype.

The naturally-occurring allelic combinations (“haplotypes”) of these 11 ns-SNPs encode 14 structurally-distinct ADAMTS13 proteins. The domain structures of the 14 structurally-distinct forms (designated here as haplotypes 1 through 14), which are encoded by the naturally-occurring allelic combinations of these 11 ns-SNPs are shown in FIG. 2B. The 14 haplotpes are made up of the following combinations of amino acids at positions 7, 448, 456, 458, 625, 740, 900, 982, 1033 and 1226 in the ADAMTS13 protein: H1 (RQQPPREQGQT) (SEQ ID NO: 1; H2 (REQPPREAGAT) (SEQ ID NO:2); H3 (RQQPPREVGAT) (SEQ ID NO: 3); H4 (WQQPPREAGTT) (SEQ ID NO: 4); H5 (RQQPPHEVGAT) (SEQ ID NO: 5); H6 (RQHPPRKVGAT (SEQ ID NO: 6); H7 (RQQPPREAGAI) (SEQ ID NO: 7); H8 (RQQPPHEAGAT) (SEQ ID NO: 8); H9 (WQQPPREVGAT) (SEQ ID NO; 9); H10 (RQHPPRKAGAT) (SEQ ID NO: 10); H11 (WQQPPHEAGAT); H12 (RQQPPREARAT) (SEQ ID NO: 12); H13 (RQQLPREVGAT) (SEQ ID NO: 13); and H14 (WEQPAREVGAT) (SEQ ID NO: 14). Each of the 14 ns-SNP haplotypes may encode a normal allelic variant of the ADAMTS13 protein (i.e., a wild-type allele), since the 11 ADAMTS13 ns-SNPs were found in 47 unrelated healthy individuals (24 black and 23 white), none of whom had developed TTP or other clotting disorders. Four of the ns-SNPs are predicted to have a damaging affect on ADAMTS13 activity by POLYPHEN analysis and the ADAMTS13 gene is autosomal, and as such may not manifest loss-of-function consequences (e.g. the development of TTP) when present in only a single copy (i.e., an autosomal recessive disorder).

Current technology is limited by the fact that only one allelic variant of recombinant ADAMTS13 is available. Thus, these studies identified novel, naturally-occurring alleles of human ADAMTS13. The GenBank accession number for the ADAMTS13 gene on which the ADAMTS13 haplotype sequences are based is DQ422807. The nucleic acids can be made by modification of ADAMTS13 sequence provided by GenBank accession DQ422807, for example, by site-directed mutagenesis, to provide the variants: C463T, C2105G, G2131T, C2133T, C2615G, G2637A, G2981A, C3462T, C3462T, G3707A, C3755G, G3860A, and C440T in the ADAMTS13 gene.

cDNA copies of each allele can be provided using appropriately designed primers and known PCR technology. Based on the identified allelic variations disclosed herein, vectors can be designed and constructed for recombinant expression of each of these variants proteins, or peptides thereof. Recombinant protein and peptides can be used in replacement therapy, or as an antigen for the development of haplotype specific antibodies. As described below, genotyping and haplotyping can be used for determination of any patient's allelic type and correct allelic matching of ADAMTS13 for recipients of blood products, organ transplants, or future replacement ADAMTS13 products (see below) in order to prevent and treat macro- and/or microvascular thrombotic disorders. By matching these alleles to the background alleles of the patient at-risk, this approach will contribute to solving the problem that arises with the generation of antibodies that inhibit successful treatment of patients undergoing receipt of foreign products.

B. Pooled Plasma/blood

Disclosed is a pooled blood plasma product obtained by detecting a haplotype in an ADAMTS13 gene of a blood/plasma donor and placing blood/blood plasma of the blood plasma donor in an appropriate pool based on the results. Also disclosed is a method of blood plasma pooling using ADAMTS13 haplotypes. Blood plasma pooling is described generally below.

Human blood plasma is the yellow, protein-rich fluid that suspends the cellular components of whole blood, that is, the red blood cells, white blood cells and platelets. Plasma enables many housekeeping and other specialized bodily functions. In blood plasma, the most prevalent protein is albumin, approximately 32 to 35 grams per liter, which helps to maintain osmotic balance of the blood. Blood plasma is generally accumulated in two ways: plasma separated from donor collected whole blood, and from donated plasma, a process where whole blood is drawn from a donor, the plasma is separated (plasmapheresis) and then the remainder, less the plasma, is returned to the donor. Plasma pooling facilitates the treatment, for purposes of economies of scale, handling, distribution and blood safety, of collected blood plasma. This collected and aggregated blood plasma is placed in a common vat for this process. The process, produces what is known as Solvent Detergent Blood Plasma (SD plasma, PLAS+SD). SD blood plasma is a blood product that has undergone treatment with the solvent tri-N-butyl phosphate (TNBP) and the detergent Triton X-100 to destroy any lipid bound viruses including: HIV1 and 2, HCV, HBV and HTLVI and H The process does not destroy non-enveloped viruses such as parvovirus, hepatitis A virus, or any of the prion particles. The SD process includes the pooling of up to 500,000 units of thawed Fresh Frozen Blood Plasma (FFP), treating it with the solvent and detergent. The treated blood plasma pool is then sterile filtered (and thus leukocyte-reduced) before being repackaged into 200 mL aliquots or bags and re-frozen. This separation into smaller units is to facilitate handling, distribution and use by the transfusion recipient or the blood product reprocessor. SD Blood plasma can be stored for up to one year frozen at −18° C. When ordered for transfusion it is thawed in a water bath to a use temperature of 37° C., which takes approximately 25 to 30 minutes and can be kept refrigerated for up to 24 hours at 1° to 6° centigrade. Only ABO identical or compatible SD Blood plasma can be transfused.

Blood/plasma pooled according to the methods disclosed herein provide blood/plasma pools homogenous or enriched in the H1, H2, H3, H4, H5, H6, H7, H8, H9, H10, H11, H12, H13 or H14 of ADAMTS13.

### II. Methods of Use

A. Method for Transplant/Transfusion Product Matching

One of the main problems that arise with exposure to structurally-distinct (i.e., “mismatched”) therapeutic proteins, such as ADAMTS13 alleles from blood product transfusion, organ transplantation, or replacement ADAMTS13 products (both plasma-derived and recombinant) is that patients mount an alloimmune response against naturally-occurring but foreign (to one's own immune system) ADAMTS13 proteins. This occurs if one or more allelic variants of ADAMTS13 represent proteins that are not recognized as self by a patient's immune system. Any patient who is exposed to an ADAMTS13 allele that is different from their endogenous (i.e., self) protein(s) may mount an alloimmune response against the naturally-occurring variant(s) at sites of mismatched ns-SNPs and perhaps at sites other than ns-SNPs due to somatic hyper-mutation and epitope spreading (which, as described below, can lead to autoantibodies). The resulting alloantibodies then inhibit the activity (and efficacy) of foreign ADAMTS13 molecules and increase the likelihood of developing thrombotic macro- and/or microvascular disease. In addition, continued or repeat exposure to structurally-mismatched “foreign” ADAMTS13 proteins may stimulate the immune system to inadvertently produce autoantibodies against self ADAMTS13 proteins (likely through somatic hyper-mutation and epitope spreading), which result in even a greater decrease in ADAMTS13 activity and an increased likelihood of thrombus development.

Similar clinical scenarios where continued exposure to alloantigens can result in autoimmunization with autoantibody development include cases of either patients with (1) Post-Transfusion Purpura (PTP) who develop autoantibodies against “self” transmembrane glycoproteins on the surface of their own platelets after transfusion of donor platelet concentrates and exposure to a foreign platelet antigen(s), (2) mild or moderate HA with (or without) alloantibody inhibitors to infused wild-type exogenous FVIII molecules who, with continued FVIII replacement therapy, develop autoantibody inhibitors against their own endogenous FVIII and become severely affected, and (3) patients with chronic hemolytic disorders such as sickle cell disease who, with continued transfusions of allogeneic RBCs, form autoantibodies to self antigens on their own RBCs and develop an even more severe anemia.

1. Individualized Pharmacogenetic Approach

A pharmacogenetic approach is provided for the accurate prediction of alloimmunogenicity of protein therapeutics (e.g., for replacement therapy) in individual patients. Using the example of FVIII in the treatment of HA, a pharmacogenetic approach is described to calculate a patient-specific alloimmunogenicity score for each protein therapeutic. Recombinant protein-drugs are mostly “self”. They can, however, differ from the endogenous protein that confers tolerance in two important ways: 1) mutations in the endogenous protein that render it defective and 2) the occurrence of nonsynonymous single-nucleotide polymorphisms (ns-SNPs). Both mutations and ns-SNPs can result in the protein sequence of the drug-product differing from the endogenous FVIII T-cell epitopes presented in the course of thymic maturation and (immune system) education through clonal deletion of auto-reactive T lymphocytes. These differences can cause alloimmunogenicity.

While it is well established that the nature of the mutation in the patient's FVIII gene (F8) is a good predictor of the frequency of alloimmunogenicity inhibitor development (Graw J, et al. Nat Rev Genet 6:488-501 (2005)), there have been few attempts to study the effects of ns-SNPs on alloimmunogenicity despite the fact that SNPs are by far the most common source of genetic variation in the human population (Frazer K A, et al. Nature 449:851-61 (2007)). A recent clinical study did demonstrate the presence of several ns-SNPs in F8 that result in primary amino acid sequence mismatches between the infused FVIII and the endogenous FVIII protein of some but not all patients with HA (Viel K R, et al. N Engl J Med 360:1618-27 (2009)). Significant differences in the frequency of inhibitor development between patients of white-European and black-African descent may be traced to distinct population-specific distributions of these ns-SNPs (Viel K R, et al. Blood 109:3713-24 (2007)).

Importantly, a sequence mismatch between the endogenous (tolerizing) peptides and those derived from the infused protein-drug is a necessary but not sufficient condition for eliciting an immune (alloimmune) response. Large numbers of peptide fragments are released but only about 2% of all the fragments have stereochemical characteristics that allow them to fit into the binding groove of any given MHC-class-II (MHC-II) molecule in the human leukocyte antigen (HLA) system.

A critical determinant for T-cell-dependent alloimmunization to an infused protein (e.g., a therapeutic protein) is the strength at which any foreign (“non-self”) peptide(s) derived from it (i.e., the potential T-cell epitopes) bind to one or more of the distinct MHC-II molecules on the surface of an individual patient's antigen-presenting cells (APCs) (Lazarski C A, et al. Immunity 23:29-40 (2005)). Concomitant to individual and population differences in the endogenous FVIII sequence, MHC-II proteins are extremely polymorphic and their distributions also exhibit clear racial and ethnic differences (Meyer D, et al. Genetics 173:2121-42 (2005)). Thus, in terms of actual frequency of inhibitor development within a population, a non-self peptide that binds with very high affinity to an MHC-II molecule that occurs at a low overall frequency will not, by itself, result in a high frequency of FVIII inhibitor formation (and vice versa).

Due to these considerations, methods for determining the immunogenicity of an infused protein are disclosed that are based on individualized pharmacogenetic parameters. Examples of parameters for this method are shown in FIG. 11. The disclosed method can be hierarchical and based on both the type and amount of data available for each individual patient.

In some embodiments, the method involves identifying one or more epitopes in the therapeutic protein, i.e., one or more sites at which the therapeutic protein differs from the sequence of the endogenous protein. In some embodiments, the one or more epitopes are identified by determining sequence variation between the therapeutic protein and an individual's endogenous protein in the subject, wherein an amino acid fragment having the sequence variation in the therapeutic protein is an epitope for the subject

In preferred other embodiments, the subject's endogenous protein sequence is identified by determining the nucleic acid sequencing of the gene encoding the endogenous protein in the subject. This step can involve sequencing a nucleic acid sample from the subject that encodes the endogenous protein. Alternatively, this step can involve screening a nucleic acid sample from the subject for specific mutations or polymorphisms. For example, this method can involve the use of primers or probes (e.g., on an array) to identify SNPs in the DNA encoding the endogenous protein. For example, the method can involve screening for specific sequence SNPs or other variations known to bind MHC-II molecules.

Null mutations that result in a loss of protein expression are cross-reacting material negative (“CRM−”). However, some mutations that result in a loss of protein in the subject's plasma demonstrate intracellular synthesis. Therefore, the CRM status in intracellular compartments is the relevant predictor for immunogenicity. For example, only about one in five HA patients having the 1221 mutation in F8, which results in no detectable protein in the plasma of patients, actually develop inhibitor antibodies. That is because the inversion results in the synthesis of the entire FVIII sequence, albeit as two polypeptide chains, thus providing tolerance to the infused FVIII protein. These patients can be tolerant to the endogenous sequence of the FVIII protein as all peptides capable of being generated from the linear wild-type FVIII protein should also be generated in an 1221 patient. The only peptides to which the patient lacks tolerance is the amino acids encoded by the exon-22/exon-23 junction sequence. If one assumes a 9 amino acid binding core for MHC Class II alleles, the peptides from the infused FVIII that would be foreign to an 1221 patient would be: GNSTGTLMV (SEQ ID NO:15), NSTGTLMVF (SEQ ID NO:16), STGTLMVFF (SEQ ID NO:17), TGTLMVFFG (SEQ ID NO:18), GTLMVFFGN (SEQ ID NO:19), TLMVFFGNV (SEQ ID NO:20), LMVFFGNVD (SEQ ID NO:21), and MVFFGNVDS (SEQ ID NO:22) (amino acids 2124 and 2125 which constitute the exon-22/exon-23 junction, are in bold and underlined font, respectively).

Therefore, in some embodiments, the subject's endogenous protein sequence is identified by determining the effect of nucleic acid sequence on intracellular expression of the endogenous protein. For example, the intracellular protein expression can be determined by immunoassay. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The method can further involve identifying the MHC-II molecules present on the cells in the individual. In some embodiments, this step involves sequencing the individual's DNA encoding the MHC-II molecules. In other embodiments, the method involves screening the subject for specific MHC-II molecules, e.g., using primers or probes (e.g., on an array) to identify SNPs in the DNA encoding the MHC-II molecules. For example, the method can involve screening for specific MHC-II molecules that occur at high frequency. In other embodiments, the method involves identifying the MHC-II molecules that occur in the subject's racial or ethnic subpopulation.

The method can further involve predicting the binding affinity of the one or more sites that differ from the endogenous sequence to MHC-II molecules. This step can comprise in silico computational methods. Recent computational advances now allow reasonably accurate in silico predictions of binding affinities of peptides to specific MHC-II molecules (Wang P, et al. PLoS Comput Biol 2008; 4:e1000048). In particular, combining predictions obtained by top performing, unrelated computational algorithms has been shown to increase prediction accuracy (Wang P, et al. PLoS Comput Biol 2008; 4:e1000048). For example, in the disclosed Examples, the method makes use of a “consensus” method that predicts binding in terms of percentile rank, with a low percentile rank reflecting high affinity. In silico programs for determining MHC-II binding predictions are publically available via the Immune Epitope Database & Analysis Resource web-site (http://tools.immuneepitope.org/analyze/html/mhc_II_binding.html). This program provides six MHC class II binding prediction methods (i.e., Consensus method, Average relative binding (arb), combinatorial library, NN-align (netMHCII-2.2), SMM-align (netMHCII-1.1), and Sturniolo) for predicting MHC-II binding affinity. Generally, a percentile rank is generated by comparing the peptide's score against the scores of five million random 15 mers selected from SWISSPROT database. A small numbered percentile rank indicates high affinity. The median percentile rank of the four methods is then used to generate the rank for consensus method.

The method can further involve determining the concentration of the MHC-II molecules on the cells of the subject. In these embodiments, the presence of an epitope that binds with high affinity to MHC-II molecules that are expressed at high concentration on the cells in the subject is an indication that the infused protein is immunogenic in that subject. Similarly, the presence of an epitope that binds with high affinity to MHC-II molecules that are expressed at low concentration on the cells in the subject is an indication that the infused protein may not be immunogenic in that subject. The concentration of MHC-II molecules on the cells of the subject is preferably determined by immunoassay or by nucleic acid detection methods (e.g., RT-PCT). In other embodiments, the concentration is the average concentration of the MHC-II molecule on cells in the subject's population or subpopulation.

The method can further involve computing an immunogenicity score based on the predicted binding affinity of the therapeutic protein epitopes with one or more MHC-II molecules on the subject's cells. The immunogenicity score can also factor in the MHC-II concentration on the subject's cells. Preferably, this score is computed using the individual's specific MHC-II genotype data. A patient-specific immunogenicity score would be the most accurate as the proteins comprising MHC-II molecules are among the most polymorphic encoded by the human genome and yet each patient's APCs contain, at most, 12 distinct MHC-II molecules (i.e., four each of HLA-DR, -DQ, and -DP). As such, each patient (with the exception of identical twins) contains a unique MHC-II peptide-antigen presentation repertoire that represents a very limited portion of the enormous diversity that exists in this system at the population level. In other embodiments, such as where these data are not known and are not able to be determined, the immunogenicity score can be weighted based on MHC-II (HLA) frequencies in the whole population or within racial or ethnic subpopulations. The immunogenicity score can be weighted based on the average concentration of the MHC-II molecule in that population.

Thus, a method of predicting the immunogenicity of a thereapeutic protein in a subject involves 1) identifying one or more epitopes in the therapeutic protein; 2) identifying the MHC-II molecules present on the cells in the subject; and 3) determining the binding affinity of each epitope to the MHC-II molecules on cells in the subject. In this method, the presence of an epitope that binds with high affinity to MHC-II molecules on the cells in the subject (preferably present at high concentrations) is an indication that the therapeutic protein is immunogenic in the subject. This method can be used to select a therapeutic protein from a library of possible proteins for use in treating the subject.

A method of selecting a protein for replacement therapy in a subject involves predicting the immunogenicity of each candidate therapeutic protein using the disclosed methods, and selecting a candidate protein for use in replacement therapy in the subject that has the fewest epitopes (preferably none) that bind with high affinity to the MHC-II molecules on cells in the subject.

In some embodiments, the immunogenicity of the candidate therapeutic proteins (or an epitope from the peptide) can be confirmed in vitro. For example, the patient's own peripheral blood monocytic cells (“PBMCs”) can be used to determine whether the protein stimulates a T-cell response.

Also provided are improved protein replacement therapy methods. The methods involve administering a protein selected using the pharmacogenetic approach described above to a subject in need thereof. In other embodiments, the method involves identifying one or more alloimmunogenic epitopes in the therapeutic protein available for replacement therepy and inducing tolerization of the on or more epitopes in the subject. In some embodiments, tolerization is induced by vaccinating the subject with a peptide containing one or more epitopes. For example, methods for tolerizing a subject, such as an infant subject, is provided that involves administering a peptide containing one or more epitopes to the infant. The peptide can be co-administered with one or more immunosuppressants.

As an example, a method of treating a subject, such as an infant subject, in need of protein replacement therapy with a therapeutic protein is provided. This method can involve identifying one or more epitopes in the therapeutic protein; identifying the MHC-II molecules present on the cells in the subject; determining the binding affinity of each epitope to the MHC-II molecules on cells in the subject; identifying one or more immunogenic epitopes in the thereapeutic protein that bind with high affinity to MHC-II molecules on the cells in the subject; and vaccinating the subject with a therapeutically effective amount of one or more peptides comprising the one or more immunogenic epitopes.

Also provided is a method predicting the immunogenicity of FVIII protein in a subject with an intron-22 inversion (1221) in the F8 gene. This method can involve identifying the MHC-II molecules present on the cells in the subject and determining the binding affinity of a peptide having the amino acids encoded by the exon-22/exon-23 junction sequence in the F8 gene to the MHC-II molecules on cells in the subject. In this method, binding of the peptide with high affinity to the MHC-II molecules on the cells in the subject is an indication that FVIII protein is immunogenic in the subject. For example, the method can involve determining the binding affinity of a peptide having the amino acid GNSTGTLMV (SEQ ID NO:15), NSTGTLMVF (SEQ ID NO:16), STGTLMVFF (SEQ ID NO:17), TGTLMVFFG (SEQ ID NO:18), GTLMVFFGN (SEQ ID NO:19), TLMVFFGNV (SEQ ID NO:20), LMVFFGNVD (SEQ ID NO:21), or MVFFGNVDS (SEQ ID NO:22) to the MHC-II molecules on cells in the subject.

Also provided is a method of treating hemophilia in a subject, such as an infant subject, with an intron-22 inversion (1221) in the F8 gene. The method can involve predicting the immunogenicity of FVIII protein in the subject, and vaccinating the subject with a therapeutically effective amount of one or more peptides containing the amino acids encoded by the exon-22/exon-23 junction sequence in the F8 gene. For example, the peptide can contain a segment having the amino acid sequence GNSTGTLMV (SEQ ID NO:15), NSTGTLMVF (SEQ ID NO:16), STGTLMVFF (SEQ ID NO:17), TGTLMVFFG (SEQ ID NO:18), GTLMVFFGN (SEQ ID NO:19), TLMVFFGNV (SEQ ID NO:20), LMVFFGNVD (SEQ ID NO:21), or MVFFGNVDS (SEQ ID NO:22).

2. ADAMTS13

Since any one subject can express at most only two of these ADAMTS13 proteins, it is believed that red blood cell transfusion to a subset of patients with a condition such as Sickle cell disease (SCD) allows exposure to different

ADAMTS13 haplotypes to which they are not immunologically-tolerant. Consequently, these patients develop alloantibodies (and in some cases autoantibodies) that tip the balance in favor of insufficient ADAMTS13 activity, and increased levels of ultra-large VWF multimers. In the case of SCD, increased levels of ultra-large VWF multimers lead to a greater propensity for painful sickle cell crises, resulting in increased hospitalization and decreased quality of life. In addition, since the less-frequent, racially-restricted alleles of four ns-SNPs (R7W, P458L, P618A, and G982R) define six of the 14 haplotypes of ADAMTS13, i.e. 4, 9, 11, 12, 13, and 14 (FIG. 2) and are predicted by POLYPHEN to encode residues that are “damaging” to the function of this protease (FIG. 1), these genetic differences alone could explain the differences in clinical severity between patients with SCD.

B. Methods for Identifying Haplotypes

1. Genotyping

Based upon the allelic variants, specific genetic test can be designed to establish the genotype and, where necessary, the haplotype of any individual using standard methodologies for SNP analysis. Methods that can be used for SNP genotyping include Rapid-cycle polymerase chain reaction (PCR) with an allele-specific fluorescent probe, High-resolution amplicon melting curve analysis or Fluorescent resonance energy transfer (FRET) hybridization probes for detection of the base changes (Lyon Molecular Diagnosis 1998 3:203, herein incorporated by reference).

A method for determining a subject haplotype can combine a rapid-cycle polymerase chain reaction (PCR) with an allele-specific fluorescent probe melting for mutation detection. This method combined with rapid DNA extraction, can generally provide results within 60 min after receiving a blood sample. This method allows for easy, reliable, and rapid detection of a polymorphism, and is suitable for typing both small and large numbers of DNA samples. The LightCycler® system enables the detection of single nucleotide polymorphisms. It combines PCR amplification and detection into a single step. The platform enables the real-time detection of a specific PCR product followed by melting curve analysis of hybridization probes. The technology is based on the detection of two adjacent oligonucleotide probes, whose fluorescent labels communicate through fluorescence resonance energy transfer (FRET). The molecular concept of single nucleotide polymorphism (SNP) detection is as follows: one of the probes serves as a tightly bound anchor probe and the adjacent sensor probe spans the region of sequence variation. During the melting of the final PCR product, the sequence alteration is detected as a change in the melting temperature of the sensor probe. For a typical homozygous wild type sample, a single melting peak is observed; for mixed alleles, two peaks are observed; and for a homozygous mutated sample, a single peak at a temperature different from the wild type allele is observed. The temperature shift induced by one mismatched base is usually between 5 and 9° C. and easily observable.

High-resolution melting of small PCR amplicons (<50 bp) is simple, rapid, and inexpensive method for SNP genotyping. Engineered plasmids representing all of the possible SNP base changes, and samples containing the medically important factor VL 5 (Leiden) 1691 G>A, prothrombin 20210G>A, methylenetetrahydrofolate reductase 1298A>C, hemochromatosis 187C>G, and /3-globin (hemoglobin S) 17A>T were successfully genotyped using this method (Liew, Clin Chem 2004 50:7), incorporated herein by reference. In all cases, heterozygotes were easily identified because the heteroduplexes altered the shape of the melting curves. Approximately 84% of human SNPs involve a base exchange between A:T and G:C base pairs (Venter Science 2001 291:1304), and the homozygotes are easily genotyped by Tms that differ by 0.8 to 1.4° C. However in the remaining SNPs₅ the bases only switch strands and preserve the base pair, producing very small Tm differences between homozygotes (<0.4° C.). Although most of these cases can still be genotyped by Tm, about a quarter have nearest neighbor symmetry (complementary 5 bases), and the homozygotes cannot be distinguished. In these cases adding a known homozygous genotype to unknown samples allows melting curve separation of all three genotypes. This method was used to identify C/C and G/G homozygotes in the hemachromatosis 187C>G SNP genotyping assay mentioned above (Liew Clin Chem 2004).

The ADAMTS13 haplotyping assay allows the rapid detection and genotyping of non-synonymous single nucleotide polymorphisms (nsSNPs), for example, of the C to T at mRNA position 1463, C to G at mRNA position 2105, G to T at mRNA position 2131, C to T at mRNA position 2133, C to G at mRNA position 2615, G to A at mRNA position 2637, G to A at mRNA position 2981, C to T at mRNA position 3462, G to A at mRNA position 3707, C to G at mRNA position 3755, G to A at mRNA position 3860, and C to T at mRNA position 4440, from DNA isolated from human whole peripheral blood. The test can be performed on the LightCycler® Instrument utilizing polymerase chain reaction (PCR) for the amplification of ADAMTS13 DNA recovered from clinical samples and fluorigenic target-specific hybridization for the detection and genotyping of the amplified ADAMTS13 DNA. The ADAMTS13 haplotyping test is an in vitro diagnostic test for the detection and genotyping of twelve non-synonymous human ADAMTS13 SNPs. The ADAMTS13 test will aid physicians in selecting matched ADAMTS13 replacement products that reduce the frequency at which recipients develop alloantibodies and immunologic refractoriness to replacement therapy. Use of the ADAMTS13 haplotyping test as a component assay in laboratory algorithms can improve the diagnostic accuracy of vasoocclusion risk assessment, since the findings of recent genetic studies have demonstrated that the alleles of at least one of the these four nsSNPs ADAMTS13 (i.e., R7W, P458L, P618A and G982R) are predicted by POLYPHEN to encode residues that are “damaging” to the function of this protease (FIG. 1), these genetic differences alone could explain the differences in clinical severity between patients with SCD.

2. Protein Detection

A subject's haplotypes, e.g., MHC-II or ADAMTS13, may be determined by protein detection methods. For example, a subject's ADAMTS13 haplotype can also be categorized by detecting a ADAMTS13 protein and categorizing the haplotype of the ADAMTS13 as being an H1, H2, H3, H4, H5, H6, H7, H8, H9, H10, H11, H12, H13 or H14.

The method includes obtaining a biological sample from the subject and detecting the presence of any of the haplotype antigens using an appropriate ligand. Antibodies can be generated to allow for the detection of haplotype antigens. In one embodiment, the immunogen is an ADAMTS13 variant peptide containing one or more amino acid sequence changes consistent with the H1, H2, H3, H4, H5, H6, H7, H8, H9, H10, H11, H12, H13 or H14 of ADAMTS13. ADAMTS13 variant peptides are used to generate antibodies that recognize any of the ADAMTS13 haplotypes, including H1, H2, H3, H4, H5, H6, H7, H8, H9, H10, H11, H12, H13 or H14 of ADAMTS13. Such antibodies include, but are not limited to polyclonal, monoclonal, chimeric, single chain, Fab fragments, and Fab expression libraries. The term ““monoclonal antibody”” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include ““chimeric”” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies to ADAMTS13 variants corresponding to the disclosed haplotypes can be made using any procedure which produces monoclonal antibodies. For example, monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes maybe immunized in vitro, e.g., using the HIV Env-CD4-co-receptor complexes described herein. The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al. In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

Screening for the desired antibody can be accomplished by techniques known in the art (e.g., radioimmunoassay, ELISA (enzyme-linked immunosorbant assay), ““sandwich”” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

Antibody binding is detected by detecting a label on the primary antibody. The primary antibody can also detected by detecting binding of a secondary antibody or reagent to the primary antibody. The secondary antibody can be labeled. Many means are known in the art for detecting binding in an immunoassay. As is well known in the art, the immunogenic peptide should be provided free of the carrier molecule used in any immunization protocol. For example, if the peptide was conjugated to keyhole limpet hemocyanin (“KLH”), it may be conjugated to albumin, or used directly, in a screening assay.)

The antibodies can be used in methods known in the art relating to the localization and structure of ADAMTS13 (e.g., for Western blotting), measuring levels thereof in appropriate biological samples, etc. The antibodies can be used to detect ADAMTS13 H1 to H14 haplotypes in a biological sample from an individual. The biological sample can be a biological fluid, such as, but not limited to, blood, serum, plasma, interstitial fluid, urine, cerebrospinal fluid, and other fluids or tissues containing cells.

The biological samples can be tested directly for the presence of ADAMTS13 using an appropriate strategy (e.g., ELISA or radioimmunoassay) and format (e.g., microwells, dipstick (e.g., as described in WO 93/03367), etc. Alternatively, proteins in the sample can be size separated (e.g., by polyacrylamide gel electrophoresis (PAGE), in the presence or not of sodium dodecyl sulfate (SDS), and the presence of ADAMTS13 detected by immunoblotting (Western blotting). Immunoblotting techniques are generally more effective with antibodies generated against a peptide corresponding to an epitope of a protein.

C. Gene Therapy

Gene therapy is a basis for treatment of for people with severe congenital ADAMTS13 deficiency and other heritable bleeding and clotting disorders. Donor and recipient allele matching for ADAMTS13 replacement is of utmost importance at the DNA level for designing various recombinant expression vectors. The method allows each congenital ADAMTS13 deficient patient undergoing gene therapy to receive an allelically matched replacement ADAMTS13 protein. This is important because such a response in the gene therapy setting may potentially result in both neutralizing antibodies against the protein and lytic responses against host tissues that are successfully transduced with the gene therapy vector.

The nucleic acid sequences of the ADAMTS13 variants corresponding to the haplotypes disclosed herein are useful with various methods of nucleic acid delivery. For example, in a subject with a given haplotype of ADAMTS13, the nucleic acid sequence corresponding to the full length ADAMTS13 variant amino acid sequence of that haplotype can be administered to the subject, thereby increasing the amount of the proper ADAMTS13 variant in that particular subject. The nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA. Such methods are well known in the art and readily adaptable for use with the compositions and methods described herein. In certain cases, the methods will be modified to specifically function with large DNA molecules.

1. Liposomes

The compositions can comprise, in addition to the disclosed genes or vectors for example, lipids such as cationic liposomes (e.g., DOTMA, DOPE, DC-cholesterol) or anionic liposomes. Liposomes can further comprise proteins to facilitate targeting a particular cell, if desired. Liposomes are disclosed for example in Brigham, et al. Am. J. Resp. Cell. Mol. Biol. 1:95-100 (1989); Feigner et al. Proc. Natl. Acad. Sci USA 84:7413-7417 (1987); and U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Commercially available liposome preparations such as LDPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art can be used.

2. Nucleotide Vectors

Transfer vectors can be any nucleotide construction used to deliver genes into cells (e.g., a plasmid), or as part of a general strategy to deliver genes, e.g., as part of recombinant retrovirus or adenovirus.

Vector delivery can be via a viral system, such as a retroviral vector system which can package a recombinant retroviral genome (see e.g., Pastan et al., Proc. Natl. Acad. Sci. U.S.A., 85:4486, 1988; Miller et al., MoI. Cell. Biol. 6:2895, 1986). The recombinant retrovirus can then be used to infect and thereby deliver to the infected cells nucleic acid encoding an ADAMTS13 haplotype of choice. The exact method of introducing the altered nucleic acid into mammalian cells is not limited to the use of retroviral vectors. Other techniques are widely available for this procedure including the use of adenoviral vectors (Mitani et al., Hum. Gene Ther,. 5:941-948, 1994), adeno-associated viral (AAV) vectors (Goodman et al., Blood, 84:1492-1500 (1994)), lentiviral vectors (Naidini et al., Science, 272:263-267 (1996)), pseudotyped retroviral vectors (Agrawal, et al., Exper. Hematol., 24:738-747 (1996)). Physical transduction techniques can also be used, such receptor-mediated and other endocytosis mechanisms (see, for example, Schwartzenberger et al., Blood 87:472-478 (1996)). This disclosed compositions and methods can be used in conjunction with any of these or other commonly used gene transfer methods.

As used herein, plasmid or viral vectors are agents that transport the disclosed nucleic acids, such as a given haplotype of ADAMTS13 into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Merleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase in transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(i) Retroviral Vectors

A retrovirus is an animal virus belonging to the virus family of Retro viridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(ii) Adenoviral Vectors

The construction of replication-defective adenoviruses has been described (Berkner et al., J. Virology 61:1213-1220 (1987); Massie et al., MoI. Cell. Biol. 6:2872-2883 (1986); Haj-Ahmad et al., J. Virology 57:267-274 (1986); Davidson et al., J. Virology 61:1226-1239 (1987); Zhang ““Generation and identification of recombinant adenovirus by liposome-mediated transfection and PCR analysis”” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest., 92:1580-1586 (1993); Kirshenbaum, J. Clin. Invest., 92:381-387 (1993); Roessler, J. Clin. Invest., 92:1085-1092 (1993); Moullier, Nature Genetics, 4:154-159 (1993); La Salle, Science 259:988-990 (1993); Gomez-Foix, J. Biol. Chem., 267:25129-25134 (1992); Rich, Human Gene Therapy, 4:461-476 (1993); Zabner, Nature Genetics, 6:75-83 (1994); Guzman, Circulation Research, 73:1201-1207 (1993); Bout, Human Gene Therapy, 5:3-10 (1994); Zabner, Cell, 75:207-216 (1993); Caillaud, Eur. J. Neuroscience, 5:1287-1291 (1993); and Ragot, J. Gen. Virology, 74:501-507 (1993)). Recombinant adenoviruses achieve gene transduction by binding to 5 specific cell surface receptors, after which the virus is internalized by receptor-mediated endocytosis, in the same manner as wild type or replication-defective adenovirus (Chardonnet and Dales, Virology, 40:462-477 (1970); Brown and Burlingham, J. Virology, 12:386-396 (1973); Svensson and Persson, J. Virology, 55:442-449 (1985); Seth, et al., J. Virol. 51:650-655 (1984); Seth, et al., MoI. Cell. Biol., 4:1528-1533 (1984); Varga et al., J. Virology, 65:6061-6070 (1991); Wickham et al., Cell, 73:309-319 (1993)).

If the nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001 (1997); Alvarez and Curiel, Hum. Gene Ther., 8:597-613, (1997). A subject can receive a single injection, or, if additional injections are necessary, they can be repeated appropriate time intervals, as determined by the skilled practitioner) for an indefinite period and/or until the efficacy of the treatment has been established.

(iii) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site 0 specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP. In another type of AAV virus, the AAV contains a pair of inverted 25 terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(iv) Large Payload Viral Vectors

Molecular genetic experiments with large human herpesviruses have provided a means whereby large heterologous DNA fragments can be cloned, propagated and established in cells permissive for infection with herpesviruses (Sun et al., Nature, 15 genetics 8: 33-41, 1994; Cotter and Robertson,. Curr Opin MoI Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The compositions can be administered in a pharmaceutically acceptable carrier and can be delivered to the subject(s) cells in vivo. Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

### III. Kits for Analyzing ADAMTS13 Haplotype

Kits are provided for determining whether or not an individual contains any of the haplotypes H1 to H14 of ADAMTS13. In some embodiments, the kits are useful for matching donor products ADAMTS13-containing products to recipients. The diagnostic kits are produced in a variety of ways. In some embodiments, the kits contain at least one reagent for specifically detecting the H1 to H14 haplotypes. In some preferred embodiments, the kits contain reagents for detecting a SNP caused by a single nucleotide substitution of the wild-type gene. In these preferred embodiments, the reagent is a nucleic acid that hybridizes to nucleic acids containing the SNP and that does not bind to nucleic acids that do not contain the SNP. In other preferred embodiments, the reagents are primers for amplifying the region of DNA containing the SNP. In still other embodiments, the reagents are antibodies that preferentially bind either the H1 to H14 ADAMTS13 proteins. In some embodiments, the kits include ancillary reagents such as buffering agents, nucleic acid stabilizing reagents, protein stabilizing reagents, and signal producing systems (e.g., florescence generating systems as Fret systems). The test kit may be packaged in any suitable manner, typically with the elements in a single container or various containers as necessary along with a sheet of instructions for carrying out the test. In some embodiments, the kits also preferably include a positive control sample.

Although described with reference primarily to ADAMTS13, it will be understood that the same methods and reagents and kits can be used to detect and utilize other haplotypes involved in the etiology of hemophilia.

The following are examples of how these methods and reagents can be utilized.

Identification of the Causative HA Mutation and FVIII Transcript Expression

DNA, RNA, plasma, and cells can be isolated from blood. Samples can be collected in EDTA tubes for genomic DNA isolation, PAXgene tubes for RNA isolation, and heparin tubes both for immortalizing B-lymphocytes and cryopreservation of viable PBMCs.

Since the PUP studies' patients' F8 genes have been sequenced to a variable extent, but never fully, a study can be initiated by sequencing, bi-directionally, the patients' F8 genes using Sanger fluorescent sequencing methodology. A SQL database of each patient's F8 mutation(s) including those that define genotypes as well as all other single-nucleotide polymorphism (SNP) sites, can be created. The causative HA mutation in each patient can be confirmed.

FVIII protein can be quantitated using both genotype-specific and region-specific immunofluorescence assays. Plasma cross-reactive material (CRM)-status can be evaluated using ELISA and a panel of anti-FVIII antibodies to the A1, A2, A3, B, C1, and C2 domains of FVIII. Plasma from normal individuals can be used as a positive control.

HLA-II Repertoire and FVIII-Derived Peptide Binding Analysis

The most highly-variable, immunologically important region, i.e., exon-2 of HLA-II, that is expressed in the DRB1, DRB3, DRB4, DRB5, DQA1, DQB1, DPA1 and DPB1 alleles can be sequenced in each patient. Analysis of HLA-II allele sequences represents a significant challenge given both the hypervariability of HLA-II genes and the fact that, unlike the single copy of the F8 gene that can be encountered in males with HA, there can be multiple copies of the HLA-II genes.

Each patient's individual HLA-II repertoire, as it pertains to FVIII-derived peptide binding, can be assessed using software that aggregates the results of multiple computational algorithms from the Immune Epitope Database & Analysis Resource, each of which predicts HLA-II peptide binding affinities. Every possible overlapping 15-mer FVIII peptide (i.e., 1-15, 2-16, 3-17, . . . , 2318-2332) can be used to predict the binding affinity of each patient's individual HLA-II molecules. This computational methodology can be used to calculate an overall immunogenicity potential of the infused peptide for each patient.

Whether the computational analysis accurately predicts high affinity binding of FVIII-derived peptides to specific HLA-II molecules can be confirmed by having a limited number of FVIII peptides synthesized and measuring their binding affinities to HLA-II molecules. For each location where a mismatch exists between the patient's own F8 genotype and that of the infused drug, the binding of 7 peptides can be measured in vitro. These peptides can be offset from each other by two amino-acids (i.e., 0, 2, 4 and 6 amino acids in each direction from the origin, which is the mismatched amino acid). Each peptide can be tested for binding to the HLA-II alleles sequenced in the patient. These experiments can be performed using a cell-free HLA-II binding assay where binding and dissociation constants of peptide-MHC complexes will be measured using a conformational ELISA with the appropriate, purified HLA-II molecule and anti-HLA antibody.

A heuristic computational analysis, adjusting contributory weights for each piece of added information, can be constructed that creates an optimized model for inhibitor development potential based upon a retrospective analysis of subject inhibitor status. There is, therefore, a potential advantage in analyzing the PUP studies' subjects in two distinct groups. Models derived from the analyses of the first group of subjects can be tested against data obtained from the second group of subjects. At this juncture it should be noted that, as each new refinement is added to the model, it can be determined whether or not there has been an improvement in the ability to predict the development of inhibitory antibodies to the infused protein.

Further to refine the strategy assessing the interaction between FVIII-derived peptides and the immune system an assay can be developed to measure the expression of HLA-II allele-specific mRNA, quantitated by RT-PCR, in PBMC-derived total RNA. Actual HLA-II expression levels can be used to narrow the focus of the HLA-II/peptide algorithm to reflect both HLA-II expression levels in addition to binding affinity. This can predict with even greater accuracy the likelihood that immunologically-important FVIII-derived peptides will trigger an immune response.

FVIII Alternate Transcript Expression in PBMCs

The analysis can be refined even further with a determination of whether each patient might express a nascent FVIII protein, encoded as an alternate transcript(s), containing FVIII peptides that would have resulted in Th-cell deletion in utero, thereby negating their immunogenicity potential. The F8 sequence data can be complemented by measuring the expression levels of all mRNA transcripts known to contain F8 exonic sequences. In addition to F8 itself, these alternate transcripts include F8_{FT}, F8_{B}, as well as several other recently-identified transcripts.

EBV-immortalized B-cells from each patient can be stained with the same panel of anti-FVIII antibodies and intracellular FVIII levels determined using flow cytometry and confocal microscopy to assess the potential for synthesis of nascent F8 gene-derived proteins that fail to be translocated outside the cell. This is referred to as “intracellular cross-reactive material” (intracellular CRM). Non-permeabilized cells and isotype control antibodies (in place of FVIII antibodies) can be used as negative controls

Using PBMC-derived RNA and quantitative RT-PCR, the impact of each HA-causing mutation on expression levels of all transcripts known to contain F8 exonic sequence, including F8, F8_{FT}, F8_{B}, and a few recently identified putative alternative transcripts, can be characterized.

FVIII-Derived Peptides' Stimulation of T-Cell Proliferation

Following the successful demonstration of high affinity binding of FVIII-derived peptides to HLA-II molecules, it can be determined whether these same peptides stimulate a T-cell response using the patient's own PBMCs. Peptides identified as potential T-cell epitopes can be used in T-cell proliferation assays with the patient's own PBMCs. This can be used to determine whether a quantitative difference in the number of molecules of each HLA-II allele expressed on the surface of PBMCs influences the immunogenicity of the same replacement FVIII protein in patients with the same mutation (e.g., the intron 22 inversion) and the same pre-mutation ns-SNP-based genotype.

The present invention will be further understood by reference to the following non-limiting examples.

### EXAMPLES

### Example 1

### Factor VIII (FVIII) in Hemophilia A (HA) patients with the Intron-22 Inversion (1221): Implications for FVIII Tolerance and Immunogenicity

Materials and Methods

Human subjects and tissue preparation: The lymphoblastoid cells used in this study were derived from a normal individual and a HA patient with the 1221.

Cell: Human lymphoblastoid cell lines developed from a severe HA patient with the 1221 and a normal control were cultured in RPMI with 10% heat inactivated fetal bovine serum, 1% penicillin-streptomycin, 1% glutamine at 37° C. with humidified 5% CO₂ incubator.

Flow Cytometry: Cells were grown overnight in complete RPMI, harvested, fixed and permeabilized the according to the manufacture's instructions (IntraPrep™, Beckman Coulter, Marseille, France). Unpermeabilized cells were used as control. Monoclonal antibodies against different domains of the human factor VIII were used for labeling. Anti-mouse IgG2a served as negative controls. The primary antibodies were detected using an Alexa Fluor 488-labeled goat anti-mouse IgG secondary antibody. The staining was performed at 37° C. for 30 min followed by three washes with 0.2% bovine serum albumin (BSA; Sigma USA) in PBS (pH 7.4). Cells were then analyzed using Becton Dickinson FACS caliber and median value of fluorescence intensity was determined using the Cell Quest software (Becton Dickinson, USA).

Confocal Microscopy: Cells were grown overnight in complete RPMI supplemented with 10% fetal bovine serum. Cells were harvested next day followed by three times wash with Phosphate buffer saline supplemented with 0.2% BSA. Fixation was performed by 4% paraformaldehyde (PFA, EMS Inc, USA) for 20 min at RT followed by permeabilization with 0.2% triton −100× (Sigma, USA) for 5 min at RT. Factor VIII protein was labeled using monoclonal antibodies against N-terminal region of the 83 kD light chain (ab4l 188; abcam Inc, MA, USA) and C2 domain of light chain (ESH-8, American Diagnostic, Inc, USA) of human factor VIII for one hour at RT followed by one hour incubation with secondary anti-mouse detection antibody conjugated with Alex fluor 488 at RT. In a co-localization study of FVIII protein within cell organelles, cells were labeled with rabbit polyclonal antibody against anti-human GRP78/BiP for ER (ab21685, Abcam Inc, USA), LAMP1 for lysosomes (ab24170, Abcam Inc, USA) and Giantin for Golgi bodies (ab24586, Abcam Inc, USA) for overnight at 4° C. after one hour labeling with FVIII protein. The Alexa Fluor 488 conjugated anti-mouse IgG (Invitrogen, USA) and Texas Red conjugated anti-rabbit IgG (ab6800, Abcam, Inc, USA) secondary antibodies were used for detection. Nuclear counter staining was performed with Vectashield mounting medium with DAPI (Vector Lab, USA). Labeling with Secondary antibody only served as a control. Confocal Images were acquired with Zeiss AM software on a Zeiss LSM 510 Confocal microscope System (Carl Zeiss Inc, Thornwood, N.Y.) with a Zeiss axiovert 100M inverted microscope.

Knockdown of FVIII protein with SiRNA: FVIII protein expression in Lymphoblast cells were knocked down using Smart Pool F VIII targeted SiRNA Dharmacom, USA)) at a concentration of 1-4 μM for 1×10⁶/ml in Accel medium using Accel delivery system (Dharmacom, USA) as per manufacture's protocol. The control cells were transected with non target-scrambled SiRNA pool at a final concentration of Glucose 6 Phosphate dehydrogenase (GAPDH) targeted SiRNA was used as internal control. Cells were harvested 72 hours post transfection and immuno-stained for the flow cytometry using above anti-human Factor VIII antibodies.

Results

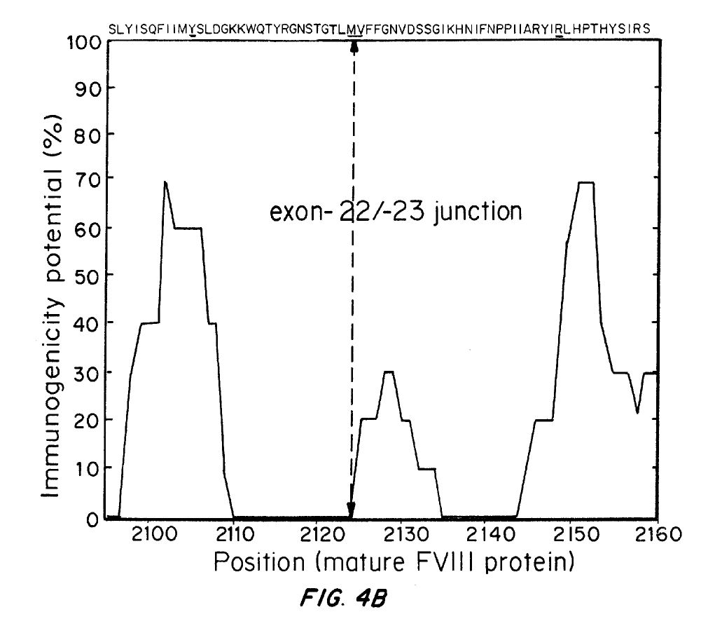

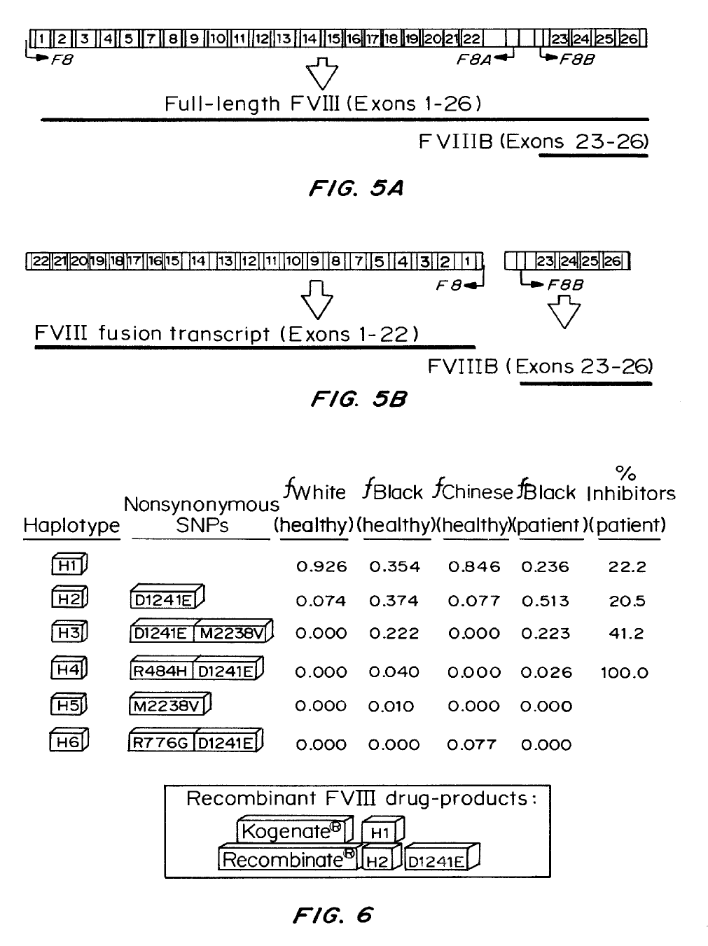

Although the infusion of Factor VIII (FVIII) to Hemophilia A (HA) patients is a preeminent example of the successful management of a chronic disease, the development of inhibitory antibodies in ˜20% of patients is currently the most significant impediment to this strategy. With improvements in technology and the increased use of recombinant FVIII; product related risk-factors for immunogenicity have been minimized. Clinical studies have provided evidence that genetic factors, particularly the nature of FVIII gene (F8) mutations, are determinants of individual responses vis-à-vis immunogenicity. Synthesis of the FVIII polypeptide chain is necessary for inducing central tolerance; thus for example while HA patients with missense mutations in F8 develop inhibitors with a frequency of about 5%, the rate of inhibitor development for patients with large gene deletions has been reported to be as high as 88%. Interestingly, this precept does not appear to apply to the 1221 mutation, which occurs in about half of all severe HA patients. This large alteration in F8 results in no detectable protein in the plasma of patients. However, only about one in five HA patients with the I22I mutation actually develop inhibitor antibodies. Based on the F8 gene structure (FIG. 5a), it is possible for the entire primary sequence of the FVIII protein to be synthesized by patients with the I22I. The intron-22 (I22) of the 188 kb F8 gene contains two nested genes, F8_{A} and F8_{B}, the transcription of which is regulated by a shared bi-directional promoter. The structure of F8 in individuals with I22I illustrates that transcription of the inverted F8 locus yields a polyadenylated fusion transcript (FT), F8_{FT}, that contains FVIII exons 1-22 (FIG. 5b).

As shown in FIG. 5a, the 186 kb F8 gene consists of 26 exons. Intron 22 (I22) contains two nested genes (F8A and F8B). The spliced F8 mRNA is approximately 9 kb in length and translated into a precursor protein of 2,351 amino acids. The F8_{B} mRNA is also translated into the FVIII_{B} protein.

As shown in FIG. 5b, a fragment (referred to as int22h1) within intron 22 of the F8 gene has sequence similarities to two fragments that are distal to the F8 gene (int22h2 and int22h3). By intrachromosomal homologous recombination, one of these outside regions forms a crossing-over structure with the corresponding element within intron 22, resulting in an inversion of exons 1-22 with respect to exons 23-26 of the F8 gene. Thus as a consequence of the I22I, a polypeptide FVIII_{FT} is synthesized which encompasses exons 1-22 of the wild type protein. Moreover, due to the position of the nested gene FVIII_{B} polypeptide coded by exons 23-26 of the wild-type F8 gene can also be synthesized. As depicted, together the FVIII_{FT} and the FVIII_{B} incorporate the entire primary sequence of the wild type protein.

The intron-22 inverted locus also encodes two polyadenylated mRNAs containing the F8 exonic sequence, F8 fusion transcript and F8_{B}. The F8_{B} mRNA and FVIII_{B} protein it encodes are identical to that encoded by the wild-type locus. The 5′-end of the fusion transcript is comprised of F8 exons 1-22 while its 3′-end contains at least 551 bases of non-F8 sequence from the extended portion of the duplication located closest to the telomere of Xq. This non-F8 3′-end sequence is incorporated by RNA Pol II transcription of genomic DNA adjacent to exon-22 in the rearranged locus followed by splicing of at least two intronic segments. While two non-F8 exons were detected, additional exons may reside 3′ to them. These could not be seen because of the priming site of the reverse transcriptase oligonucleotide used in the one study that characterized the mRNA from nucleated blood cells of inversion patients. Translation of this mRNA is predicted to yield a polypeptide that contains the entire amino acid sequence encoded by F8 exons 1-22 (i.e., residues −19 to −1 of the primary translation product and 1 to 2124 of the mature circulating FVIII protein) fused at its C-terminus to 16 non-F8 residues.

Moreover, due to the location of the int22h-1 and the nature of homologous recombination, there should be complete synthesis of the wild-type F8_{B} gene. Together the polypeptides synthesized from the F8_{FT} and F8_{B} transcripts would contain the entire primary sequence for the full-length FVIII protein.

Materials and Methods

To study FVIII expression, mRNA levels were estimated in immortalized lymphoblastoid cells obtained from a normal individual and a HA patient with the 1221. Three sets of forward and reverse primers that probed the regions of exons 1-22, exons 23-26 and the exon-22/exon-23 junction were used. Relative quantification of F8 mRNA levels in the two cells was performed using housekeeping gene, GAPDH.

Intracellular expression of proteins can be identified by antibody staining followed by flow cytometry. To detect the full-length FVIII as well as the FVIII_{FT} and the FVIII_{B} polypeptide chains the antibodies ESH4, ESH5, ESH8, and Ab41188 were used to target the C2, A1, C2, and A3 domains of the FVIII protein. Permeabilized cells from a normal individual and an HA patient with the I22I labeled with the secondary antibody alone, anti-FVIII antibodies Ab-41188 which detects the A3 domain, and ESH8 which detects the C3 domain. The secondary antibody was conjugated to the fluorophore, Alexa Fluor 488.

Permeabilized cells from a normal individual were co-labelled with the mouse anti-FVIII antibodies Ab-41188 and ESH8 as well as the rabbit polyclonal antibody against anti-human GRP78/BiP as ER marker, anti-human Giantin as Golgi marker, and anti-human LAMP1 as lysosomal marker. Alexa Fluor 488 conjugated anti-mouse IgG and Texas Red conjugated anti-rabbit IgG secondary antibodies were used for detection.

Permeabilized cells from an individual with the I22I were co-labelled with the mouse anti-FVIII antibodies Ab-41188 and ESH8 as well as rabbit polyclonal antibodies to detect ER, Golgi and lysosomal markers as described above.

Sections obtained from the liver that was excised from a HA patient with the I22I who received a transplant as well as from the donor liver from a normal individual were stained with mouse anti-FVIII antibodies Ab-41188 and ESH8 and detected using a secondary antibody conjugated to the fluorophore, Alexa Fluor 488.

To further demonstrate that the lymphoblastoid cells do indeed synthesize FVIII and that the antibodies used are specific, Smart Pool FVIII targeted siRNA was used to knockdown the protein. The Smart Pool siRNA specific to FVIII was used at concentrations of 1, 2 and 5 μM; scrambled siRNA (μM) was used as a negative control and siRNA targeted to GAPDH (μM) as a positive control.

Sections from a liver were obtained that was removed from a HA patient who received a liver transplant due to chronic hepatitis A and C as a result of FVIII infusions. These sections were stained with the anti-FVIII antibodies Ab41188 and ESH5.

Results

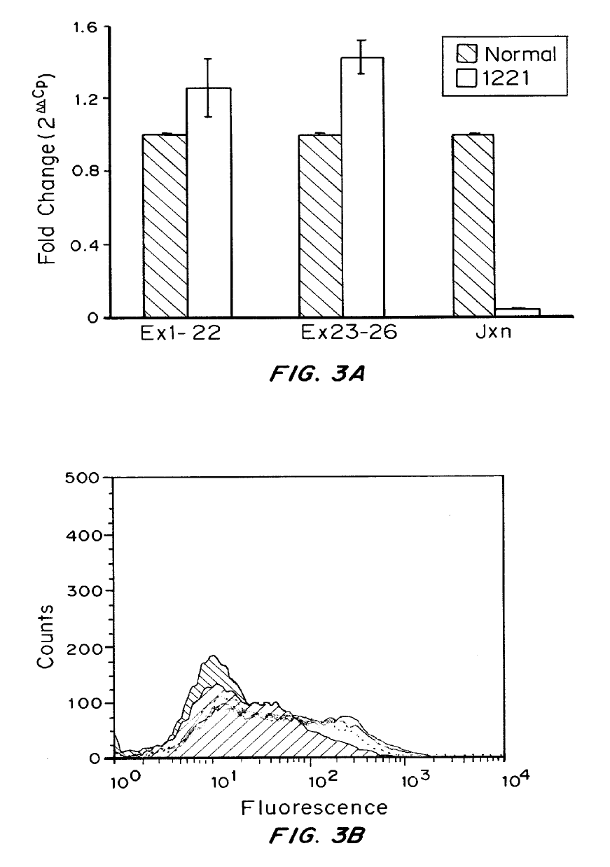

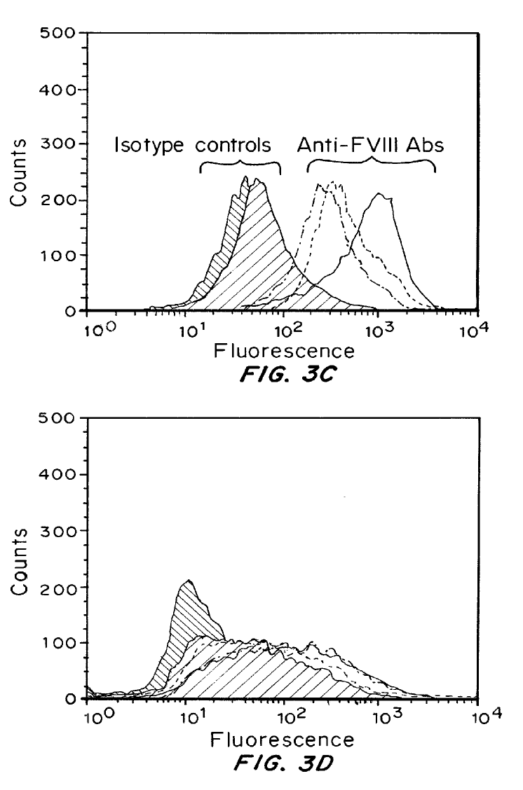

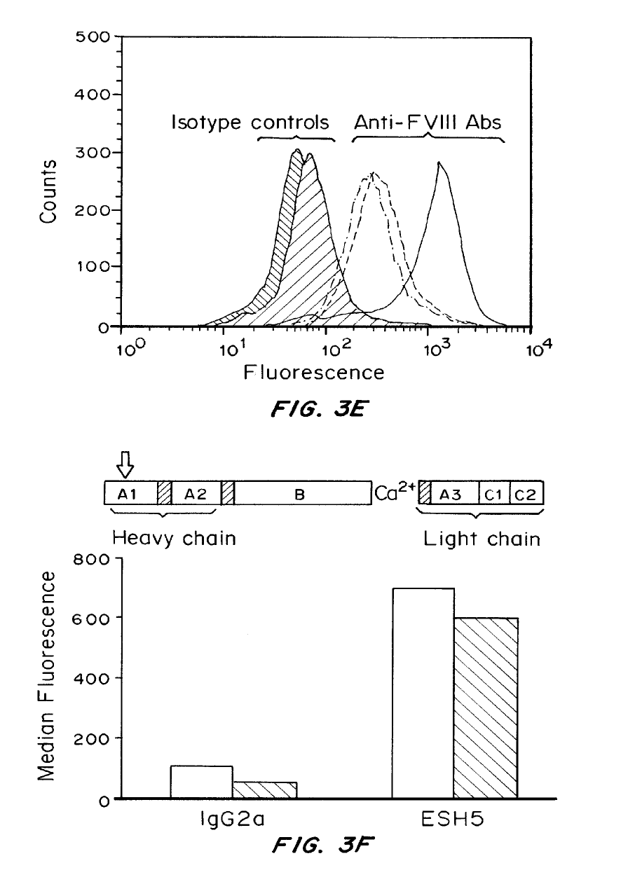

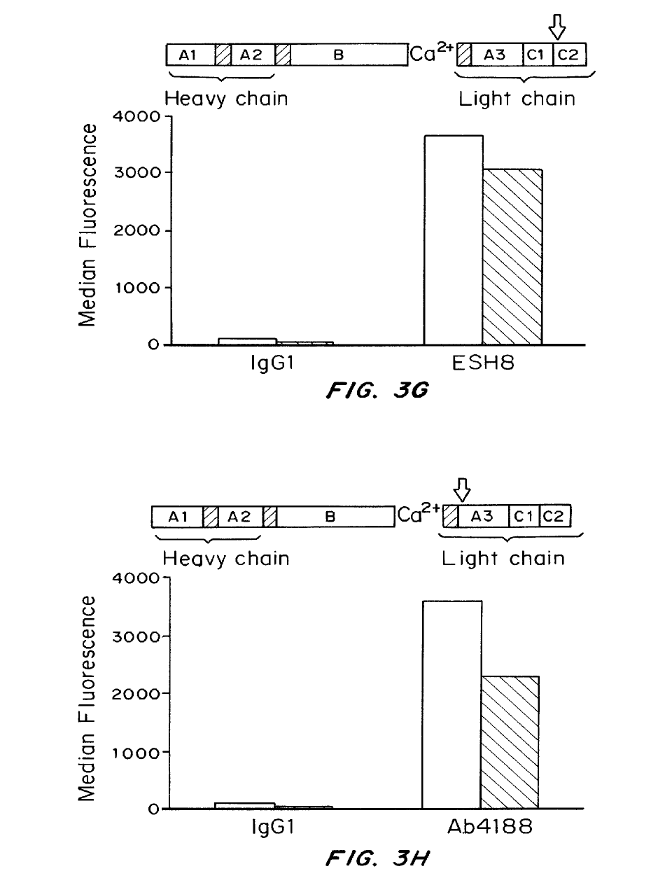

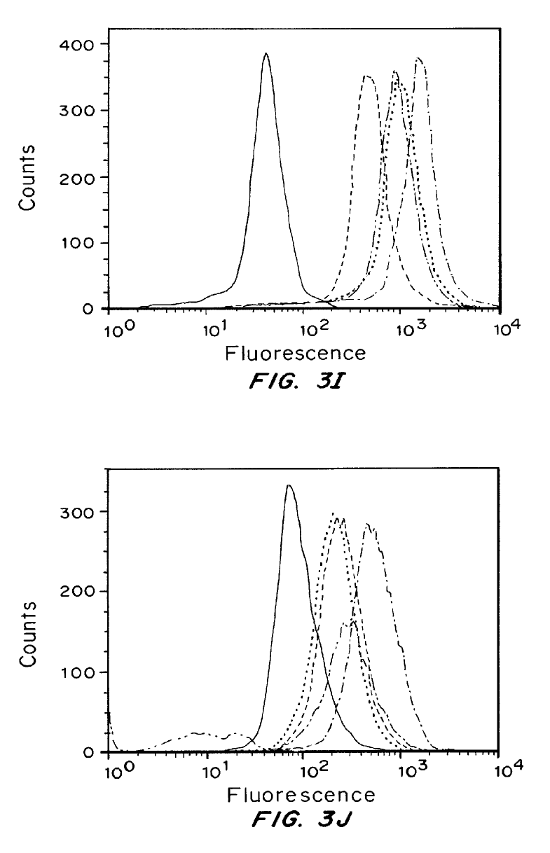

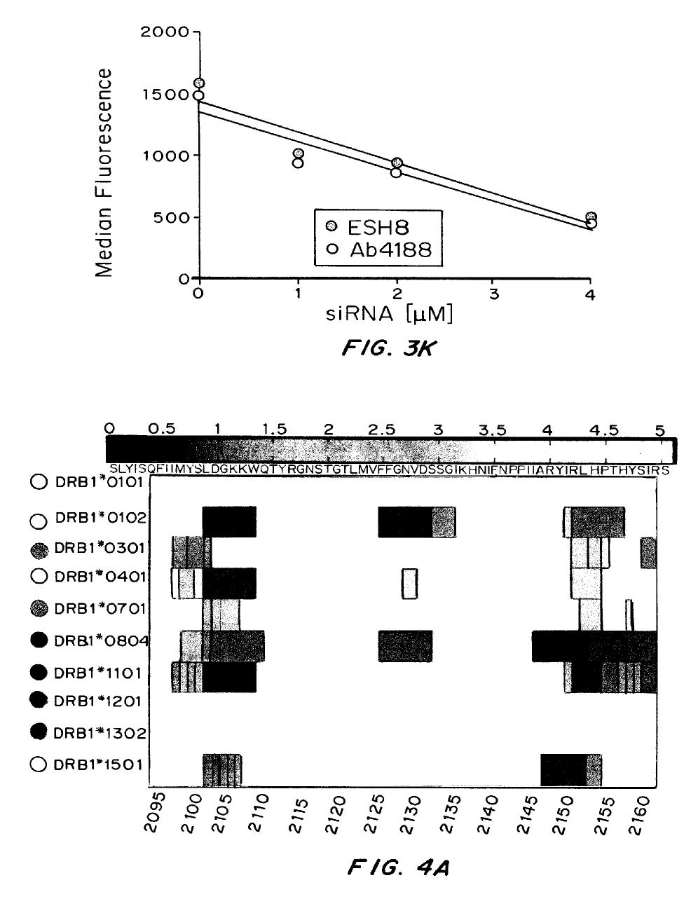

The primers that span the exon-22/exon-23 junction detected F8 mRNA in normal cells but not in cells derived from the I22I patient (FIG. 3a). On the other hand primers that detect exons 1-22 and 23-26 boundaries detected F8 mRNA in cells from both the normal individual and the I22I patient. Relative quantification shows that F8 mRNA levels in cells derived from the patient were comparable of those in normal cells (FIG. 3a).

There was a minimal shift in fluorescence of the anti-FVIII antibodies compared to the isotype control antibodies (FIG. 3b-3e) and secondary antibody alone the in non-permeabilized cells. However in permeabilized cells from the normal individual as well as the HA patient, there was a 5-30 fold increase in fluorescence intensity when anti-FVIII antibodies were used compared to the isotype control antibodies tagged with the same secondary antibody (FIG. 3b-3e). The use of the antibodies ESH5 and Ab41188 demonstrated equivalent expression of the heavy and light chains respectively in cells derived from the normal individual and the HA patient with the I22I. However the antibody ESH8 recognized amino acids 2248-2285 and could thus identify only the C2 domain of FVIII. In the normal individual the positive signal with the ESH8 antibody would detect either the full-length FVIII or the FVIII_{B}, as this antibody detects the C2 domain of FVIII. However, in the I22I patient, the larger FVIII_{FT}does not carry the C2 domain and thus the ESH8 antibody detected the FVIII_{B} polypeptide alone.

A decrease in the FVIII signal (using either the ESH8 or Ab41188 antibodies) was observed in cells transfected with FVIII specific siRNA (FIG. 3f-3h) but not in cells transfected with the scrambled siRNA. Moreover there was a linear decrease in the FVIII signal as a function of siRNA concentration (FIG. 3f-3h) and the siRNA (at the highest concentration) reduced the FVIII levels by approximately 70%. These data clearly demonstrate that the flow cytometry based method used monitors intracellular levels of FVIII. However, though this technique permits the detection of protein and relative quantification, it does not allow for sub-cellular localization of the FVIII.

Cells derived from the normal individual and the HA patient both showed a FVIII-positive labeling with the antibodies Ab41188 and ESH8 (A3 and C2 domains) when imaged using confocal microscopy. In addition co-localization studies were performed using the ER, ER and lysosomal markers, GRP78/BiP, Giantin and LAMP1 respectively. It has been extensively reported that the trafficking of FVIII is inefficient and a significant proportion of the primary translation product is targeted to the cellular degradation machinery. This is consistent with prior findings that cells from the normal patient show co-localization of FVIII with all three subcellular organelles suggesting that at least some of the FVIII is targeted lysosomal degradation. Antibodies that detect FVIII_{FT} and FVIII_{B} polypeptides in cells from the HA patient stain the FVIII polypeptides in all three organelles.

Although low-levels of FVIII were synthesized in the lymphoblastoid cells and were detected using sensitive techniques, the primary physiological site for in vivo expression remains unknown. Nonetheless most studies have determined that FVIII is at least expressed in the liver. Therefore, sections from a liver removed from a HA patient were stained with the anti-FVIII antibodies Ab41188 and ESH5. Positive staining by the anti-FVIII antibodies Ab41188 and ESH5 in liver samples from the HA patient with the I22I indicates that both the FVIII_{FT} and FVIII_{B} polypeptides were synthesized (FIG. 3).

Taken together these studies clearly demonstrate that the I22I per se does not prevent the synthesis of the FVIII protein. These patients would thus be tolerant to the endogenous sequence of the FVIII protein as all peptides capable of being generated from the linear wild-type FVIII protein should also be generated in an I22I patient. The only peptides to which the patient would lack tolerance would be the amino acids encoded by the exon-22/exon-23 junction sequence. If one assumes a 9 amino acid binding core for MHC Class II alleles, the peptides from the infused FVIII that would be foreign to an 1221 patient would be: GNSTGTLMV (SEQ ID NO:15), NSTGTLMVF (SEQ ID NO:16), STGTLMVFF (SEQ ID NO:17), TGTLMVFFG (SEQ ID NO:18), GTLMVFFGN (SEQ ID NO:19), TLMVFFGNV (SEQ ID NO:20), LMVFFGNVD (SEQ ID NO:21), and MVFFGNVDS (SEQ ID NO:22) (amino acids 2124 and 2125 which constitute the exon-22/exon-23 junction, are in bold and underlined font, respectively).

However, a mismatch between the endogenous and the infused peptide is a necessary but not a sufficient condition to elicit an immune response as less than 2% are loaded onto MHC Class II proteins. A computational assessment of this region of the FVIII protein shows that it is unlikely to immunogenic (FIG. 4). Non-synonymous (ns)-single-nucleotide polymorphisms (SNPs) in the F8 gene represent significant variations in the FVIII sequence in the human population. Moreover, a mismatch between the endogenous FVIII sequence of the patient and the infused FVIII due to the sequence variation introduced by the ns-SNPs is a significant risk factor for the development of inhibitory antibodies.

Thus in about half of all patients with severe HA, the disease causing defect, the I22I per se, has minimal or modest effect on immunogenicity and the underlying ns-SNPs represent the most important risk factor. This finding is of importance in the clinic because Caucasians exhibit very little variability vis-à-vis ns-SNPs in the F8 gene whereas individuals of African descent show significant variability. On the other hand the recombinant FVIII products match the endogenous sequence that characterizes Caucasians. Several studies have shown that there is a significant disparity in the frequency with which African American HA patients develop inhibitory antibodies compared to Caucasian patients. It is likely that underlying ns-SNPs in the patient population could also explain why the frequency of inhibitor development varies widely in different groups of patients with the I22I.

### Example 2

### Factor VIII (FVIII) Inhibitors and the Intron-22 (122) Inversion (1221): Implications for Immunologic Tolerance and Immunogenicity

Factor VIII (FVIII) inhibitors occur in approximately 20% of all treated hemophilia A (HA) patients with the prevalence being highest in those that are severely affected. The development of these neutralizing anti-FVIII antibodies is a complex process involving both treatment- and patient-related risk factors, the most striking of which is the structure of the FVIII gene (F8).

The nature of the F8 mutation causing HA strongly influences the propensity for inhibitor development. Additionally, naturally occurring non-synonymous (ns)-single-nucleotide polymorphisms (SNPs) are found in pre-mutation F8 genes in various populations forming patterns described as haplotypes 1 to 8. Haplotypes 1 and 2 are found in Caucasians and in the majority of African Americans, Chinese, and individuals from other racial groups studied thus far, as well as in the currently-licensed recombinant FVIII concentrates (FIG. 6). To date, haplotypes 3, 4, 5, 7, and 8 have been found only in African Americans; the relevant ns-SNPs are predominantly in the immunogenic A2- and C2-domains. African American HA patients whose hemophilia mutations occurred in F8 with an H3 or H4 background haplotype were found to have developed inhibitors about three times as frequently as African American HA patients with an H1 or H2 haplotype. The patients with an H3 or H4 haplotype had been transfused with one or more brands of recombinant FVIII concentrates (containing either the H1 or H2 protein) and/or plasma-derived FVIII concentrates (enriched in the H1 and H2 protein), thus they had received “mismatched” replacement therapy.

Since this mismatching can add to the risk of inhibitor formation, multiple recombinant wild-type versions of the FVIII protein should be developed in order to provide allogeneically matched products for more patients, especially for those with black African ancestry.

Pharmacogenetic Relevance of Mutations

The patients most likely to benefit from haplotype matched FVIII concentrates are those with “pharmacogenetically-relevant” F8 mutation types. This phrase is used herein to refer to HA-causing mutations that do not disrupt the transcription of any F8 exon and, in most instances, only slightly affect the amino acid sequence of FVIII. A fetus can become immunologically tolerant to their endogenous (“self”) FVIII proteins and, after birth, may tolerate structurally similar wild-type FVIII replacement products. For such a patient, a replacement product matched to the greatest extent possible to his pre-mutation FVIII structure might be the least likely to provoke an inhibitor. Missense mutations, which account for approximately 35-40% of all HA patients, represent examples of this mutation type. Inhibitors have been reported to develop in only about 5% of patients with F8 missense mutations overall, however, greater alloimmunization risk can be associated with certain sites of amino acid substitution and with the degree of biochemical difference between the side chains of the wild-type and mutant amino acid residues. The on-line database HAMSTeRS (Hemophilia A Mutations, Structure, Test and Resource Site) (http://hadb.org.uk/) shows that inhibitor development has occurred in 15-50% of patients who have one of five highly recurrent missense mutations (Arg593Cys, Tyr2105Cys, Arg2150His, Pro2300Leu, or Trp2229Cys) and 100% of patients with either Argl997Pro or Asn2286Lys, two of the less frequent recurrent mutations. Additionally, more than 50 non-recurrent inhibitor-associated missense mutations have been reported. These observations indicate that replacement proteins can induce alloantibodies even when infused in patients whose mutant endogenous FVIII proteins differ from the wild-type by as little as a single residue.

Certain null-type F8 defects are pharmacogenetically-irrelevant because they involve loss of large segments of FVIII coding sequence, which precludes the fetus from becoming tolerant to large portions of the wild-type protein. A replacement protein has little with which to be matched; all replacement proteins are likely to be equally “foreign”. With large deletions involving multiple exons, the incidence of inhibitors is greater than 65% and possibly as high as 88%. When there is genomic loss of F8 coding sequence, not only is there no plasma FVIII (i.e., cross-reacting material negative, or “CRM−”, HA) but intracellular synthesis of the full-length FVIII mRNA and polypeptide also are precluded. Such synthesis is a requirement for central tolerization of the immune system towards the antigen. Some large exonic deletions and duplications, however, occur in-frame, and thus might not prevent the resultant mutant F8 from driving synthesis of most or all of a FVIII protein, respectively, that lacks cofactor activity. With nonsense mutations, premature termination (stop) codons prevent intracellular synthesis of the full-length FVIII protein. The location of mutant stop codons may also be a determinant of inhibitor formation. Inhibitors develop in about 40% of patients with nonsense mutations in sequences encoding the FVIII light chain, but in less than 20% of patients with nonsense mutations in sequences corresponding to the FVIII heavy chain.

The intron-22 inversion (1221), which causes about 40-45% of all severe HA cases, is the most common cause of HA with CRM−plasma, and is the second most common pharmacogenetically relevant mutation type. An international survey of 2093 severe HA patients (Antonarakis, 1995) reported that only 1 in 5 patients with the 1221 had become alloimmunized after replacement therapy, a frequency less than that observed in patients with the inhibitor-associated recurrent missense mutations described above and approximately equal to that observed in general in patients with severe HA of all causes. Despite this report, I22I continues to be widely regarded as a high risk mutation for inhibitors. Propagation of this belief probably has occurred, in part, because I22I causes a CRM−plasma FVIII deficiency, analogous to patients with large F8 deletions, the highest risk null-type mutation, and, in part, because I22I is so frequent.

Intracellular CRM Status and Intron-22 Inversions

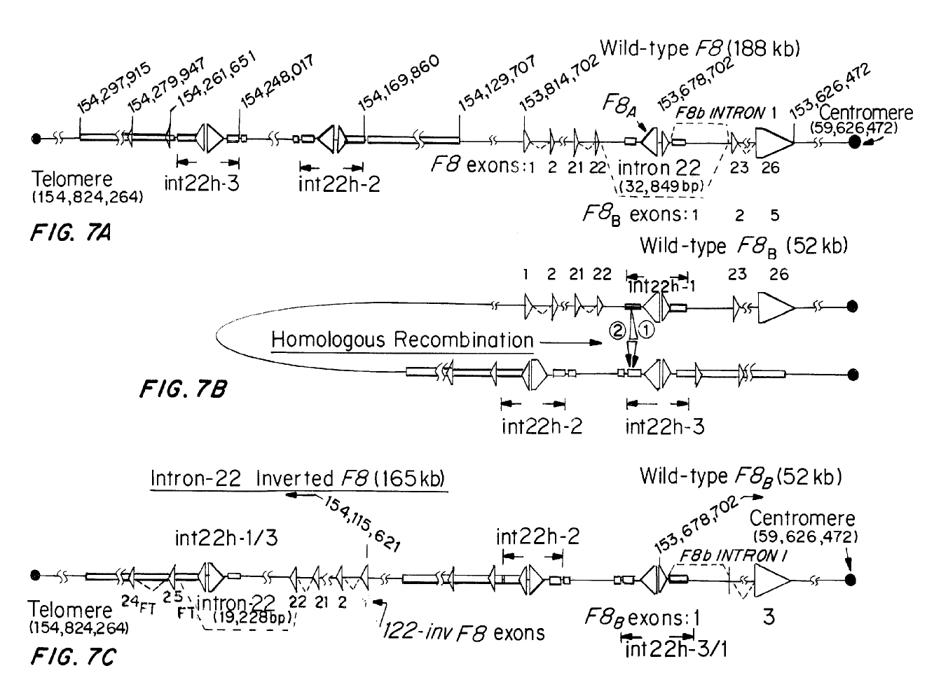

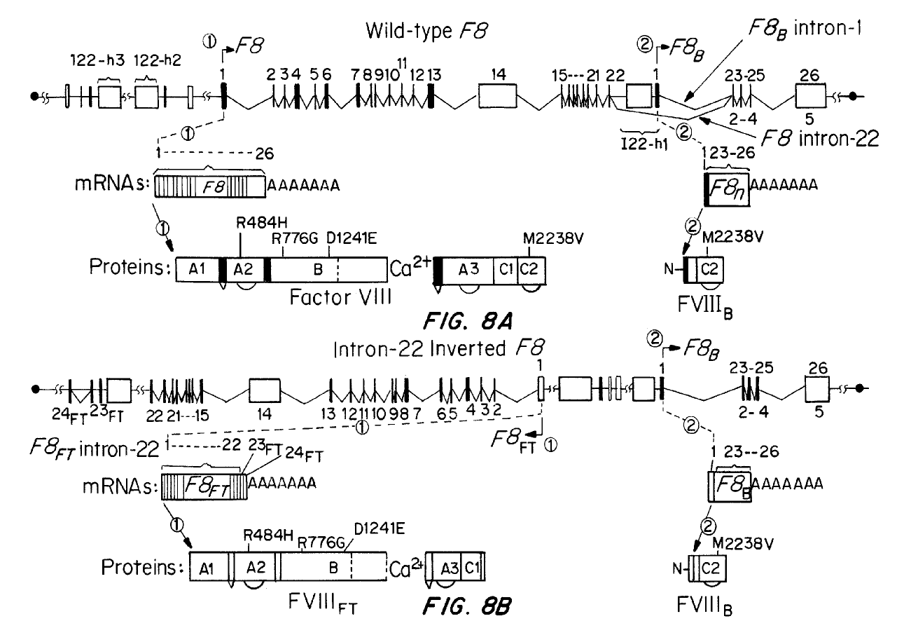

A model is provided that accounts for the lower-than-presumed incidence of inhibitors in I22I patients. The new phrase “intracellular CRM status” is used herein to categorize F8 null mutations as causing either CRM+ or CRM− intracellular FVIII deficiencies. The loss of multiple exons precludes transcription and translation of a full-length transcript and protein. Large deletions clearly cause CRM− intracellular deficiencies, thus preventing fetal induction of immunologic tolerance to FVIII or at least to any portions missing from the endogenous FVIII protein. In contrast, it is predicted that I22I causes a CRM+intracellular FVIII deficiency. A diagram is provided representing the genomic structure of the wild-type and inverted F8 alleles (FIG. 7) to explain why. As shown in the upper panel, F8 is a 188 kilobase (kb) gene located near the telomere at Xq28.1. It contains 26 centromerically-oriented exons, which, through a 9,030 base-pair (bp) polyadenylated mRNA, code for a 2,351 amino acid protein (including the 19 residue leader-peptide) (FIG. 8A). The 32,849 by intron-22 contains an approximately 9.5 kb sequence, designated int22h-1, which includes a single exon gene, F8_{A}, and exon-1 of a five exon containing gene, F8_{B}. Transcription of F8_{A} and F8_{B} is regulated by a shared bi-directional promoter. Two essentially identical sequences to int22h-1, int22h-2 and int22h-3, are located, respectively, approximately 355 kb and approximately 433 kb telomeric to F8. F8 and F8_{B} are both transcriptionally oriented towards the centromere. As shown in FIG. 7, intranemic homologous recombination between int22h-1 and int22h-3 (middle panel) results in the I22I (FIG. 7B-7C). F8_{B}'s promoter and first exon are located within int22h-1, which is centromeric of and oriented oppositely to int22h-3, thus, this rearrangement results in truncation of the wild-type F8 transcription unit (i.e., lacking exons 23-26) and inversion towards the telomere. The inversion juxtaposes exon-22 to a genomic region normally located telomeric to exon-1, which contains two cryptic exons (GenBank No. U00684) and appropriate 5′- and 3′-splice junction sequences (FIG. 7), but the F8 promoter and regulatory region are left intact.

Transcription of the inverted F8 locus followed by primary mRNA processing yields a polyadenylated fusion transcript (FT), F8_{FT}, that contains FVIII exons 1-22 spliced to these two cryptic exons, designated here as 23_{FT} and 24_{FT} (FIGS. 7C and 8B). Exon-23_{FT} contains 16 in-frame codons followed by an in-frame stop codon and 38-bp of untranslated sequence. Because this stop codon is situated less than 50-55 nucleotides upstream of the 3′-most exon-exon junction (i.e., between 23_{FT} and 24_{FT}), the fusion transcript is not predicted to trigger nonsense-mediated mRNA decay. This is consistent with results from non-quantitative, end-point RT-PCR-based assays in which the fusion transcript levels appear to be equivalent to or greater than that of the full-length, wild-type FVIII mRNA. Thus, upon translation of the fusion transcript only 16 additional amino acids are predicted to be incorporated into a fusion protein that would contain 2,159 amino acids including the 19-residue native FVIII leader-peptide (FIG. 8B). There is complete restoration of the wild-type F8_{B} gene, which encodes a widely expressed moderately abundant 2.6 kb polyadenylated transcript with exons 23-26 of F8 spliced in-frame to an unrelated first exon that has a Kozak's consensus initiation codon. The F8_{B} mRNA is predicted to code for a 216 amino acid protein containing an 8-residue N-terminal segment encoded by exon-1 followed by 208 residues encoded by exons 2-5, which, as shown in FIG. 8, correspond to exons 23-26 in F8.

F8_{FT} and F8_{B}, the two polyadenylated F8-derived transcripts found in blood cells from all patients with I22I (FIG. 8B), which together contain the entire contiguous coding sequence for the full-length FVIII protein, are transcribed and translated in the developing thymus and thus allow wild-type FVIII peptides to be generated intracellularly and presented on HLA class II molecules. The predicted expression of HLA class II proteins complexed with FVIII peptides on the surface of medullarly thymic epithelial cells—a specialized type of professional antigen presenting cell whose main function is thought to be to “educate” the T-lymphocyte component of the immune system towards self antigens through clonal deletion of auto-reactive T cells—could, with the possible small exception detailed below, result in central tolerance to the full-length wild-type FVII1 protein.

FIGS. 7 and 8A-8B show that while the inverted F8 allele cannot be transcribed into a full-length mRNA nor, therefore, translated into a full-length functional FVIII protein, as F8_{FT} lacks exons 23-26, the reconstituted F8_{B} transcription unit incorporates these remaining F8 exons into the F8_{B} mRNA. This suggests that within FVIII-producing cells of an I22I patient, including the thymic epithelial cells, these two mRNAs may be translated into two polypeptide chains, which together contain the entire primary amino acid sequence of the FVIII protein. Since the process of becoming tolerant to a self-protein requires that it first be translated, I22I patients can be tolerized to the specific form of FVIII encoded by their discontinuous F8 exonic sequences. An I22I patient may be tolerized to replacement FVIII if it is matched to the form of the protein encoded by his background F8 haplotype.

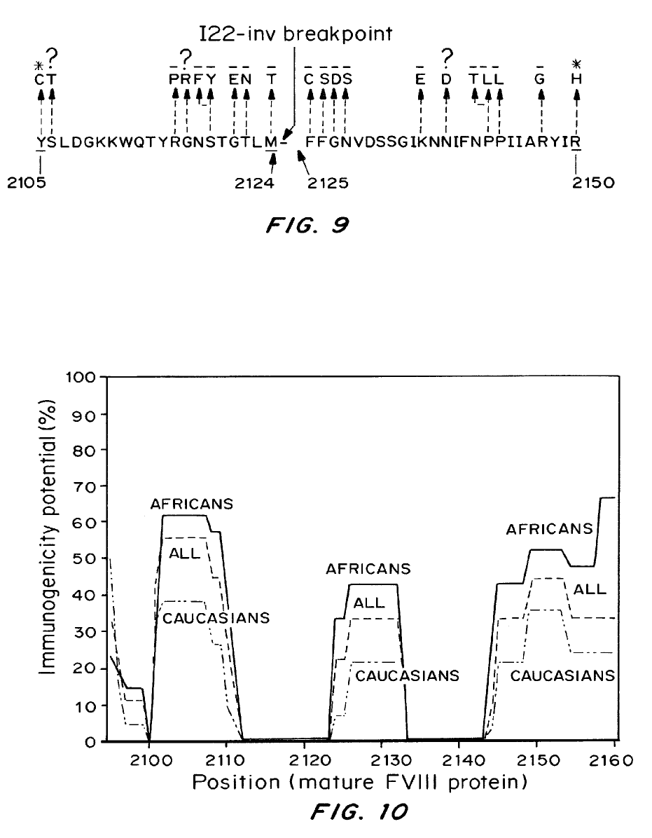

The last base of exon-22 corresponds to the third nucleotide of codon 2143, which encodes methionine at position 2124 in the mature circulating FVIII protein, while the first base of exon-23 is the first nucleotide of codon 2144, which encodes valine at the immediately adjacent residue (V2125) (FIG. 9). Thus the truncation of F8 after exon-22 does not split a codon and every FVIII amino acid residue should be expressed in I22I patients. All peptides capable of being generated from the linear wild-type FVIII protein in a non-inversion patient with a given background F8 haplotype also, theoretically, should be generated in an I22I patient with the same haplotype, except those few peptides containing amino acids encoded by the exon-22/exon-23 junction sequence. Specifically, any FVIII peptide ending at or before residue 2124, the last amino acid encoded by exon-22, or beginning at or after residue 2125, the first amino acid encoded by exon-23, should also be generated in the developing thymus of I22I patients. Furthermore, any of these peptides that are expressed on thymic cell surfaces bound to autologous HLA class II antigens theoretically would induce tolerance to themselves through apoptotic clonal deletion of auto-reactive T cells whose antigen receptors recognize as epitopes these protein/peptide complexes. Although the length of peptides that may be bound in HLA class II molecules and involved in the binding by T-cell receptors is an unsettled issue, nine residues-the core-peptide length that occupies the HLA-binding cleft-were selected to illustrate that the following eight wild-type FVIII nonamers cannot be generated from the two polypeptides predicted to be translated from the two documented F8-derived transcripts encoded by the I22-inverted locus: GNSTGTLMV (SEQ ID NO:15), NSTGTLMVF (SEQ ID NO:16), STGTLMVFF (SEQ ID NO: 17), TGTLMVFFG (SEQ ID NO:18), GTLMVFFGN (SEQ ID NO:19), TLMVFFGNV (SEQ ID NO:20), LMVFFGNVD (SEQ ID NO:21), and MVFFGNVDS (SEQ ID NO:22) (amino acids 2124 and 2125 are in bold and underlined font, respectively) (FIG. 9B).

If a patient with I22I is transfused with therapeutic FVIII concentrate and if one or more of these eight peptides can be generated intracellularly and presented in vivo by HLA class II antigens, those parts of wild-type replacement proteins that encode the exon-22/exon-23 junction sequences could theoretically provoke an alloimmune response in some or all I22I patients. If this were the case, however, one would expect to see HLA-restricted immune responses to the wild-type sequence of this site. On the contrary, neither the primary (linear) structure of these 9-mer peptides nor the secondary/tertiary (3-dimensional) structure of the corresponding region in their source wild-type FVIII replacement protein have ever been found to serve as T- or B-cell epitopes, respectively, in patients with I22I or any other HA-causing F8 mutation.

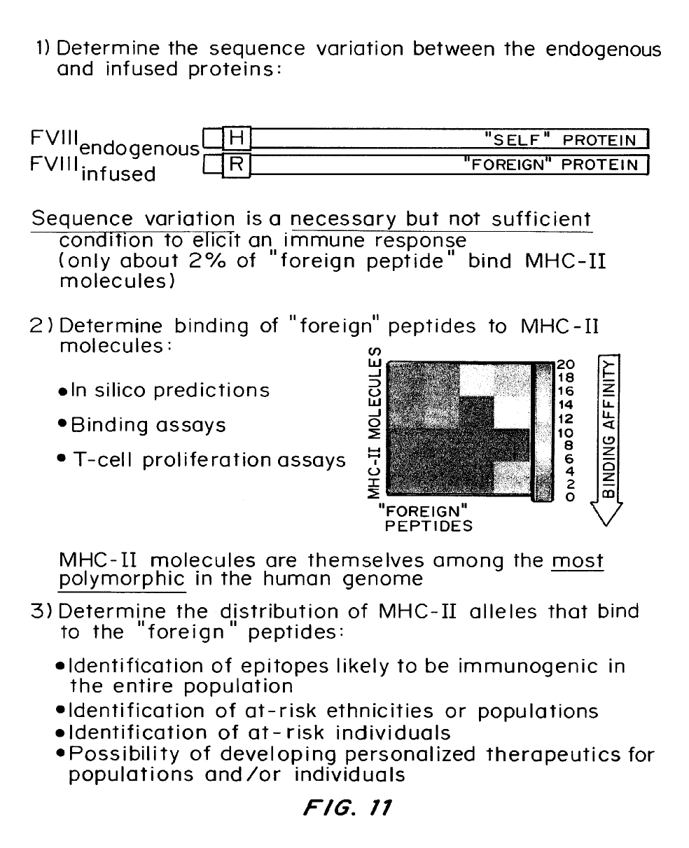

There is one exception to the self-tolerization mechanism proposed above. Because the F8-derived mRNAs transcribed from the inverted locus (FIG. 8B) are discontinuous, and a peptide length of at least nine residues is required for binding to HLA class II molecules, I22I patients would not be expected to have immune tolerance to peptides corresponding to FVIII residues 2117-2132. Therefore, exposure to such peptides following replacement therapy with FVIII could lead to antibody generation if the peptides were effectively presented on one or more class II alleles (FIGS. 9B and 10A). The exon-22/exon-23 junction region corresponds to the FVIII C1 domain, which is generally thought to be less immunogenic than the A2 and C2 domains of FVIII. Consistent with this, 18 missense mutations have been identified involving residues comprising or flanking the exon-22/exon-23 breakpoint as illustrated in the lower panel of FIG. 9. All but one of these mutations encodes non-conservative amino acid substitutions. These mutations cause mild to severe hemophilia but none has been associated with an inhibitory antibody. Furthermore, various prediction algorithms indicate that this region may be only weakly immunogenic in individuals with several of the more common class II alleles (FIG. 10). Nevertheless, helper T cells may be activated in some individuals with HLA alleles that can bind and present these peptides.

In addition, other HLA-class-II genes and their alleles can be evaluated. Their immunogenicity can be tested directly by evaluating the binding of these peptides in vitro to purified preparations of single DRB1 alleles. In complementary functional studies, the binding of these peptides could be evaluated ex vivo using peripheral blood mononuclear cells from patients with implicated HLA-class-II repertoires using either the ELIspot assay or tetramer-based analyses. These studies assess whether the T cells proliferate and secrete cytokines when stimulated with these peptides in cell culture.

To date, the human F8 gene has been found to contain four common and two less common ns-SNPs whose naturally allelic combinations encode eight distinct wild-type FVIII proteins, only two of which have the amino acid sequences found in recombinant FVIII molecules used clinically. FIG. 6A illustrates these six ns-SNPs and the eight FVIII proteins they encode. These ns-SNPs encode the following amino acid substitutions, respectively: proline for glutamine at position 334 (Q334P), histidine for arginine at position 484 (R484H), glycine for arginine at position 776 (R776G), glutamic acid for aspartic acid at position 1241 (D1241E), lysine for arginine at position 1260 (R1260K), and valine for methionine at position 2238 (M2238V). The numbering systems used to designate the positions of the amino acid substitutions encoded are based on their residue locations in the mature circulating form of wild-type FVIII. R484H and M2238V are components of the A2- and C2-domain immunodominant epitopes that include residues arginine at position 484 to isoleucine at position 508 and glutamate at position 2181 to valine at position 2243, respectively. As shown in FIG. 6B, the two full-length recombinant FVIII proteins used in replacement therapy, Kogenate (same as Helixate) and Recombinate (same as Advate), contain the same amino acid sequences found in H1 (QRRDRM, SEQ ID NO:23) and H2 (QRRERM, SEQ ID NO:24), respectively. The B-domain deleted recombinant FVIII protein, Refacto (same as Xyntha), does not contain the ns-SNP site differentiating Kogenate and Recombinate (D1241E).

As shown in FIG. 7A, F8 has 26 exons (exons 3-20, 24, and 25 are not shown), which are oriented centromerically, and is located approximately one Mb from the telomere on the long-arm of the X-chromosome. Intron-22 (122) is about 33 kb and contains an approximately 9.5 kb sequence, designated int22h-1 in FIG. 7B, that includes F8A, a single exon gene oriented telomerically, and exon-1 of a five exon, centromerically-oriented gene, F8_{B}, that shares exons 2-5 (exons 3 and 4 not shown) with F8 (exons 23-26). Two sequences homologous to int22h-1, int22h-2 and int22h-3, are located telomeric to F8. Int22h-2 and int22h-3 are each part of a larger approximately 50 kb duplication contributed primarily by an approximately 40 kb sequence. Since int22h-2 is oriented similarly, only int22h-3 undergoes direct homologous recombination with int22h-1. Int22h-2 and int22h-3 can undergo homologous recombination with each other as part of the larger 50 kb duplication. Following homologous recombination between int22h-1 and int22h-3, intra-chromosomal rearrangement results in the F8 transcription unit being truncated (i.e., lacking exons 23-26) and inverted telomerically. Due to the mechanism of homologous recombination, there is complete restoration of the wild-type F8_{B} gene and transcription unit. In both healthy individuals with wild-type F8 and severe HA patients with I22I, the F8_{B} transcript is comprised of its unique first exon, which is not found in the F8 mRNA, followed by four exons corresponding to F8 exons 23-26. The F8 inversion juxtaposes exon-22, the 3′-most exon of its truncated transcription unit, to a more telomeric genomic region that contains two cryptic exons (GenBank accession #U00684) with adequate 5′- and 3′-splice junction sequences. As such, expression of the inverted F8 locus yields a fusion transcript, F8_{FT} containing exons 1-22 spliced in-frame to these two additional exons, only the first of which is predicted to encode additional residues following the last amino acid residue of exon-22, i.e. amino acid 2124 of the mature circulating FVIII protein.

FIG. 8A shows the genomic structure of wild-type F8 and the two mRNAs containing F8 sequence, F8 (1) and F8_{B} (2). Homologous recombination between int22h-1 and int22h-3 incompletely inverts F8. Translation of F8 and F8_{B} mRNAs, respectively, yields full-length FVIII and a putative FVIII_{B} protein with unknown function. As shown in FIG. 8B, the I22-inverted F8 locus encodes two mRNAs containing F8 sequence, the F8 fusion transcript, F8_{FT} (1), and F8_{B} (2). F8_{FT} mRNA is comprised of F8 exons 1-22 fused to 551 bases of unique 3′-sequence encoded by two cryptic exons designated 23 F_{T} and 24_{FT}. Translation of F8_{FT} mRNA is predicted to yield a protein comprised of amino acids encoded by F8 exons 1-22 followed by an additional 16 non-FVIII amino acids encoded by 23 F_{T}. The FVIII_{B} protein is predicted to be identical to that expressed in healthy persons. Although no circulating FVIII antigen is detectable in I22I patients, i.e., the plasma is CRM−, it is expected that if these two proteins, FVIII_{FT} and FVIII_{B}, are expressed, then together they encompass the entire sequence of the FVIII protein.

Y2105 and R2150 are sites of recurrent missense mutations strongly associated with inhibitors. Residues 2106 to 2123 and 2126 to 2149 are two segments of C1 on either side of the I22I break-point. M2124 and V2125 are the residues flanking the inversion breakpoint. Y2105C and R2150H have been found in many alloimmunized HA patients and represent the two inhibitor-associated missense mutations closest to the exon-22/exon-23 junction (FIG. 9). Although 18 additional missense mutations have been identified in this region, none of these patients has developed inhibitors to date.

As shown in FIG. 10A, the binding affinities of nine common HLA class II proteins for peptides derived from the C1-domain region corresponding to the exon-22/-23 junction were predicted using the consensus method, publicly available via the Immune Epitope Database & Analysis Resource web-site (http://tools.immuneepitope.org/analyze/html/mhc_II_binding.html). The method assigns for each 15-mer peptide and HLA class II molecule, a percentile rank. Lower percentile ranks indicate stronger binding affinities. Peptides with percentile ranks less than two were considered to be high affinity binders. The HLA class II molecules evaluated are encoded by nine distinct DRB1 alleles, which are common in either the white European or black African populations of the USA, or in both. As shown in FIG. 10B, the immunogenicity potential for each 15-mer FVIII peptide was defined as the percent of these nine HLA class II proteins that bind with high affinity. It is important to note, that the relative frequencies of these DRB1 alleles in the two populations was not taken into account in this analysis.

### Example 3

### Pharmacogenetics and the Immunogenicity of Protein Therapeutics

Recent studies have demonstrated that T-cell epitopes play an essential role in eliciting ADAs against therapeutic proteins (Barbosa M D, et al. Clin Immunol 118:42-50 (2006). Considerable progress has also been made in the assessment of T-cell epitopes using computational, in vitro and ex vivo methods (De Groot A S, et al. Curr Opin Pharmacol8:620-6 (2008)). Unfortunately, this progress has not translated into accurate predictions of immunogenicity. Using the example of Factor VIII (FVIII) in the treatment of hemophilia A (HA), a pharmacogenetic approach, based on individual patients, is necessary for the accurate prediction of immunogenicity. In other words, in the use of most protein therapeutics, the predicament is not that all patients develop inhibitory antibodies but that some individuals, racial and/or ethnic groups, or other sub-populations have a stronger immunogenic reaction than others. Current strategies to predict immunogenicity focus largely on identifying epitopes during pre-clinical development based on the postulate that engineering such epitopes will result in a protein that is universally less immunogenic within the entire population (De Groot A S, et al. Clin Immunol 131:189-201 (2009)). Such strategies are likely to be insufficient due to the substantial genomic variability within the patient population. Thus, an alternative decision tree is disclosed that takes a personalized approach to predicting (and eventually circumventing) immunogenicity.

Recombinant protein drugs are mostly “self”. They can, however, differ from the endogenous protein that confers tolerance in two important ways. The mutations in the endogenous protein that render it defective and the occurrence of nonsynonymous (ns)-single-nucleotide polymorphisms (SNPs) can both result in the protein sequence of the drug product differing from the endogenous FVIII T-cell epitopes likely presented in the course of thymic maturation and (immune system) education through clonal deletion of auto-reactive T lymphocytes. While it is well established that the nature of the mutation in the patient's FVIII gene, F8, is a good predictor of the frequency of inhibitor development (Graw J, et al. Nat Rev Genet 6:488-501 (2005)), there have been few attempts to study the effects of ns-SNPs on immunogenicity despite the fact that SNPs are by far the most common source of genetic variation in the human population (Frazer K A, et al. Nature 449:851-61 (2007)).

A recent clinical study demonstrated the presence of several ns-SNPs in F8 that result in primary amino acid sequence mismatches between the infused FVIII and the endogenous FVIII protein of some but not all patients with HA. Significant differences in the frequency of inhibitor development between patients of white-European and black-African descent may be traced to distinct population-specific distributions of these ns-SNPs (Viel K R, et al. Blood 109:3713-24 (2007)). Importantly, a sequence mismatch between the endogenous (tolerizing) peptides and those derived from the infused protein drug is a necessary but not sufficient condition for eliciting an immune response. Large numbers of peptide fragments are released but only about 2% of all the fragments have stereochemical characteristics that allow them to fit into the binding groove of any given MHC-class-II (MHC-II) molecule in the human leukocyte antigen (HLA) system. A critical determinant for T-cell-dependent alloimmunization to an infused protein is the strength at which any foreign (“non-self”) peptide(s) derived from it (i.e., the potential T-cell epitopes) bind to one or more of the distinct MHC-II molecules on the surface of an individual patient's antigen-presenting cells (APCs) (Lazarski C A, et al. Immunity 23:29-40 (2005)). Concomitant to individual and population differences in the endogenous FVIII sequence, MHC-II proteins are extremely polymorphic and their distributions also exhibit clear racial and ethnic differences (Meyer D, et al. Genetics 173:2121-42 (2006)). Thus, in terms of actual frequency of inhibitor development within a population, a non-self peptide that binds with very high affinity to an MHC-II molecule that occurs at a low overall frequency will not, by itself, result in a high frequency of FVIII inhibitor formation (and vice versa).

Due to these considerations, methods for determining the immunogenicity of an infused protein are disclosed that are based on individualized pharmacogenetic parameters (FIG. 11). The disclosed method can be hierarchical and based on both the type and amount of data available for each individual patient. First, the site(s) at which the infused protein(s) differ from the sequence of the endogenous protein—if all or a portion(s) of one is/are produced intracellularly—can be identified. Next, an immunogenicity score can be computed based on the predicted binding affinity of each (previously studied) MHC-II molecule for the infused-protein-derived peptides spanning each mismatched position. Optimally, this score can be derived using each patient's specific MHC-II genotype data. If these data are not known and are not able to be determined, the immunogenicity score can be weighted based on HLA frequencies in the whole population or within racial or ethnic subpopulations.

A patient-specific immunogenicity score would be the most accurate as the proteins comprising MHC-II molecules are among the most polymorphic encoded by the human genome and yet each patient's APCs contain, at most, 12 distinct MHC-II molecules (i.e., four each of HLA-DR, -DQ, and -DP). As such, each patient (with the exception of identical twins) contains a unique MHC-II peptide-antigen presentation repertoire that represents a very limited portion of the enormous diversity that exists in this system at the population level. Currently, there is no database with complete genetic, molecular, immunologic, and clinical information available to comprehensively evaluate the effectiveness of the optimal strategy towards predicting alloimmune treatment outcomes. However, the Hemophilia A Mutation, Structure, Test and Resource Site (HAMSTeRS) constitutes an extensive data-base of some such information, which has been compiled from research performed over the last three decades (http://hadb.org.uk/) (Kemball-Cook G, et al. Nucleic Acids Res 26:216-9 (1998)). One important data set attempts to list all F8 missense mutations reported (by Aug. 6, 2007) either in the literature or directly to HAMSTeRS and the status of FVIII inhibitor development by the patients within which these single-base substitution mutations were identified Akin to the ns-SNPs, endogenous FVIII protein sequences carrying deleterious amino acid substitutions encoded by missense mutations provide a localized example of self versus non-self peptides with respect to the infused protein drug.

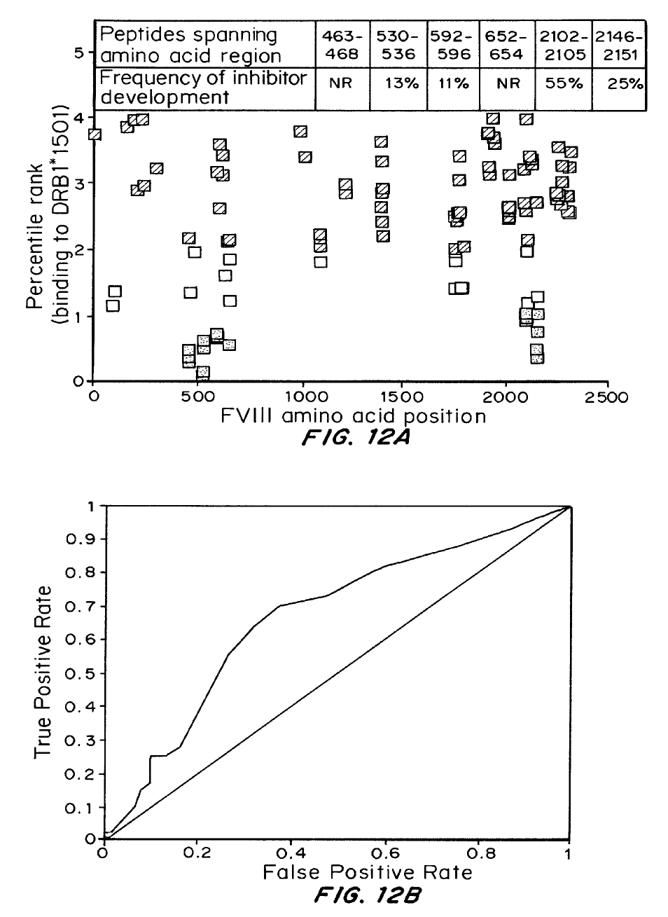

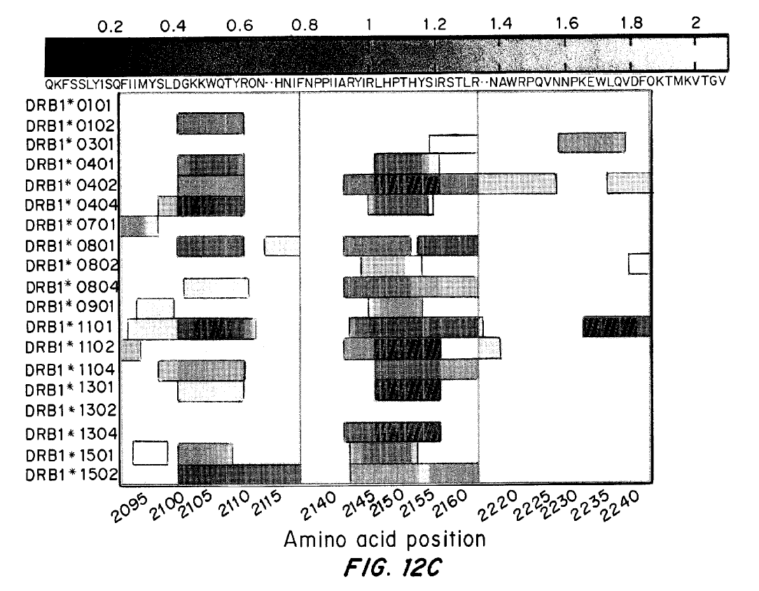

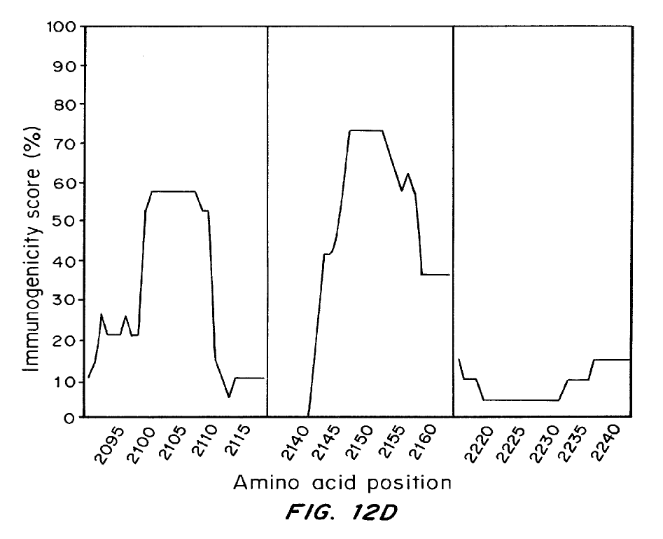

Recent computational advances now allow reasonably accurate in silico predictions of binding affinities of peptides to specific MHC-II molecules (Wang P, et al. PLoS Comput Biol 2008; 4:e1000048). In particular, combining predictions obtained by top performing, unrelated computational algorithms has been shown to increase prediction accuracy (Wang P, et al. PLoS Comput Biol 2008; 4:e1000048). The disclosed method makes use of such a “consensus” method, which predicts binding in terms of percentile rank, with a low percentile rank reflecting high affinity. FIG. 12a illustrates the predicted percentile ranks for overlapping peptides spanning the entire FVIII sequence—corresponding to the most commonly observed wild-type form of the protein in humans, referred to as haplotype 1 to HLA-DRB1*1501, an MHC-II molecule very frequently found in the human population and, particularly in white individuals with-European ancestry (who are likely overrepresented in the HAMSTeRS data-base). Only the peptides predicted to bind this MHC-II molecule are depicted (low to intermediate, high, and very high affinity binding peptides are shown).

Only a few sets (six) of overlapping peptides bind DRB1*1501 with very high affinity (see inset). Missense mutations in all of these regions are associated with mild or moderate HA and patients with such mutations in four of these regions develop inhibitory antibodies at a higher frequency than that observed in patients with this type of mutation overall (approximately 5%) (Graw J, et al. Nat Rev Genet 6:488-501 (2005)). Moreover, the regions identified as potentially immunogenic include those that encompass the amino acid positions Y2105 and 82150, which correspond to sites of highly recurrent missense mutations (Y2105C and R2150H) that are the most frequently found in patients with this F8 mutation type and inhibitor development (Oldenburg J, et al. Hemophilia 12 Suppl 6:15-22 (2006)). While anecdotal, this analysis indicates a strategy for estimating the immunogenicity of mutations at a specific position, based on the predicted binding affinities of peptides spanning that position to a relevant set of MHC-II molecules.

To more rigorously test the correlation between MHC-II/peptide binding and immunogenicity, a more global analysis of the data available in the HAMSTeRS database was performed. All sites with HA-causing missense mutations were considered. A position was labeled “positive” if at least one patient with a mutation at that position was reported to have developed inhibitors, and “negative” otherwise (i.e., no patients with a mutation at that site developed inhibitors). At each of these FVIII positions an immunogenicity score was computed, based on the number of MHC-II molecules that bind the corresponding wild-type peptides with high affinity (percentile rank <2). These immunogenicity scores significantly discriminate between positive and negative positions (area under the ROC curve=0.66; Mann-Whitney U p-value=0.0086) (FIG. 12b). Note that the HAMSTeRS data used for segregating HA-causing missense mutations into those that are or are not associated with an immunogenic response to infused FVIII is qualitative and collated over almost three decades from numerous laboratories; thus, far better discrimination would be expected in controlled studies. In addition, the availability of each patient's HLA genotyping data would allow refinement of the immunogenicity score by focusing on the much smaller set of relevant MHC-II molecules. The potential effect of incorporating information about specific HLA alleles is vividly illustrated in FIGS. 12c and 12d. The heat map depicts affinities of individual MHC-II molecules to wild-type peptides from regions of FVIII with the three highly recurrent HA-causing missense mutations (Y2105C, R2150H, and W2229C) most often found in patients that developed inhibitors. Peptides that incorporate Y2105 and R2150 show high affinity (low percentile binding rank) for most MHC-II molecules. On the other hand, peptides that incorporate W2229 appear not to bind most MHC-II molecules, however, the heat map shows that these peptides do bind with very high affinity to the MHC-II molecule HLA-DRB1*0301. A relatively high proportion of HA patients with the missense mutation W2229C develop FVIII inhibitors (33% compared to 5% overall) and the explanation for this may lie in the fact that HLA-DRB1*0301 is extremely common in the human population.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A purified or isolated haplotype of ADAMTS13 nucleic acid molecule comprising a nonsynonymous SNP.

2. The haplotype of claim 1 wherein the nonsynonymous SNP is selected from the group consisting of C463T, C2105G, G2131T, C2133T, C2615G, G2637A, G2981A, C3462T, C3462T, G3707A, C3755G, G3860A, and C440T.

3. The haplotype of ADAMTS13 of claim 2, wherein the nonsynonymous SNP encoding an ADAMTS13 protein is selected from the group consisting of H1, H2, H3, H4, H5, H6, H7, H8, H9, H10, H11, H12, H13 and H14.

4. A method of categorizing a haplotype in an ADAMTS13 gene comprising:
(a) amplifying regions of the ADAMTS13 gene;
(b) determining a haplotype of the ADAMTS13 gene from DNA sequence within the amplified regions; and
(c) categorizing the haplotype as being an H1, H2, H3, H4, H5, H6, H7, H8, H9, H10, H11, H12, H13 or H14.

5. A method of administering a blood or tissue product to a subject in need of comprising:
(a) determining which type of blood product the recipient should receive based on the haplotype of the blood product recipient; and
(b) prescribing for or administering to the subject in need thereof an appropriate blood product of the same haplotype, or a nucleic acid sequence encoding the blood product of the same haplotype.

6. The method of claim 5 wherein the blood type is an ADAMTS13 haplotype selected from the group consisting of H1, H2, H3, H4, H5, H6, H7, H8, H9, H10, H11, H12, H13 or H14.

7. The method of claim 6 wherein the ADAMTS13 haplotype has a C3755G gene variation.

8. The method of claim 5, wherein the blood product is pooled blood plasma derived from more than one blood donor.

9. A method of predicting the immunogenicity of a therapeutic protein in a subject, comprising
(a) identifying one or more potential T cell epitopes in the therapeutic protein that are foreign to the patient being infused;
(b) identifying the MHC-II molecules present on the cells in the subject; and
(c) determining the binding affinity of each epitope to the MHC-II molecules on cells in the subject;
wherein the presence of an epitope that binds with high affinity to MHC-II molecules on the cells in the subject is an indication that the therapeutic protein is immunogenic in the subject.

10. The method of claim 9, wherein the one or more epitopes are identified by determining sequence variation between the therapeutic protein and an endogenous protein in the subject, wherein an amino acid a peptide fragment comprising the amino acid sequence variation in the therapeutic protein is an epitope for the subject.

11. The method of claim 9, wherein the subject's endogenous protein sequence is identified by determining effect of nucleic acid sequence on intracellular expression of the endogenous protein.

12. The method of claim 11, wherein the intracellular protein expression is determined by immunoassay or in silico.

13. The method of claim 9, wherein the binding affinity of each epitope to MHC-II molecules is determined in silico.

14. The method of claim 9, wherein the MHC-II molecules present on the cells in the subject are identified by genotyping the subject's MHC-II haplotype.

15. The method of claim 9, wherein the MHC-II molecules present on the cells in the subject are identified by determining the MHC-II frequencies in the subject's racial or ethnic subpopulation.

16. The method of claim 9, further comprising determining the concentration of the MHC-II molecules on the cell, wherein the presence of an epitope that binds with high affinity to MHC-II molecules that are expressed at high concentration on the cells in the subject is an indication that the therapeuticinfused protein is immunogenic in that subject.

17. A method of selecting a protein for replacement therapy in a subject, comprising
(a) predicting the immunogenicity of each candidate therapeutic protein using the method of claim 9, and
(b) selecting a candidate protein for use in replacement therapy in the subject having the fewest epitopes that do not have an epitope that binds with high affinity to the MHC-II molecules on cells in the subject.

18. A method of treating an subject in need of protein replacement therapy with a therapeutic protein, comprising
vaccinating the subject with one or more peptides comprising one or more immunogenic epitopes,
wherein the epitopes are identified in the therapeutic protein;
the MHC-II molecules present on the cells in the subject are identified;
the binding affinity of each epitope to the MHC-II molecules on cells in the subject is determined; and
the one or more immunogenic epitopes in the thereapeutic protein that bind with high affinity to MHC-II molecules on the cells in the subject are determined.

19. The method of claim 18, wherein the one or more peptides are administered to the subject with in combination with immunosuppressant therapy.

20. A method of treating hemophilia in an infant subject with an intron-22 inversion comprising
vaccinating the infant subject with one or more peptides comprising the amino acids encoded by the exon-22/exon-23 junction sequence in the F8 gene in combination with
immunosuppressants, when the child is not ill or subject to immunostimulation, or via an oral, nasal or subcutaneous route, in an amount effective to induce tolerance.

21. A method of predicting the immunogenicity of a FVIII protein in a subject with an intron-22 inversion (I22I) in the F8 gene, comprising
(a) identifying the MHC-II molecules present on the cells in the subject;
(b) determining the binding affinity of a peptide comprising the amino acids encoded by the exon-22/exon-23 junction sequence in the F8 gene to the MHC-II molecules on antigen-presenting cells (APCs) in the subject;
(c) determining the binding affinity of any other foreign FVIII peptides, which can be derived from the intracellular degradation of the wild-type replacement FVIII protein at sites corresponding to ns-SNPs that are mismatched with the patient's own mutant endogenous FVIII protein, to the MHC-II molecules on APCs in the subject
wherein binding of the foreign peptide(s) with high affinity to the MHC-II molecules on the cells in the subject is an indication that FVIII protein is immunogenic in the subject.


SEQUENCE LISTING (26 sequences)

SEQ ID NO: 1 - protein, 11 aa, Artificial Sequence
  Synthetic Construct
RQQPPREQGQT

SEQ ID NO: 2 - protein, 11 aa, Artificial Sequence
  Synthetic Construct
REQPPREAGAT

SEQ ID NO: 3 - protein, 11 aa, Artificial Sequence
  Synthetic Construct
RQQPPREVGAT

SEQ ID NO: 4 - protein, 11 aa, Artificial Sequence
  Synthetic Construct
WQQPPREAGTT

SEQ ID NO: 5 - protein, 11 aa, Artificial Sequence
  Synthetic Construct
RQQPPHEVGAT

SEQ ID NO: 6 - protein, 11 aa, Artificial Sequence
  Synthetic Construct
RQHPPRKVGAT

SEQ ID NO: 7 - protein, 11 aa, Artificial Sequence
  Synthetic Construct
RQQPPREAGAI

SEQ ID NO: 8 - protein, 11 aa, Artificial Sequence
  Synthetic Construct
RQQPPHEAGAT

SEQ ID NO: 9 - protein, 11 aa, Artificial Sequence
  Synthetic Construct
WQQPPREVGAT

SEQ ID NO: 10 - protein, 11 aa, Artificial Sequence
  Synthetic Construct
RQHPPRKAGAT

SEQ ID NO: 11 - protein, 11 aa, Artificial Sequence
  Synthetic Construct
WQQPPHEAGAT

SEQ ID NO: 12 - protein, 11 aa, Artificial Sequence
  Synthetic Construct
RQQPPREARAT

SEQ ID NO: 13 - protein, 11 aa, Artificial Sequence
  Synthetic Construct
RQQLPREVGAT

SEQ ID NO: 14 - protein, 11 aa, Artificial Sequence
  Synthetic Construct
WEQPAREVGAT

SEQ ID NO: 15 - protein, 9 aa, Artificial Sequence
  Synthetic Construct
GNSTGTLMV

SEQ ID NO: 16 - protein, 9 aa, Artificial Sequence
  Synthetic Construct
NSTGTLMVF

SEQ ID NO: 17 - protein, 9 aa, Artificial Sequence
  Synthetic Construct
STGTLMVFF

SEQ ID NO: 18 - protein, 9 aa, Artificial Sequence
  Synthetic Construct
TGTLMVFFG

SEQ ID NO: 19 - protein, 9 aa, Artificial Sequence
  Synthetic Construct
GTLMVFFGN

SEQ ID NO: 20 - protein, 9 aa, Artificial Sequence
  Synthetic Construct
TLMVFFGNV

SEQ ID NO: 21 - protein, 9 aa, Artificial Sequence
  Synthetic Construct
LMVFFGNVD

SEQ ID NO: 22 - protein, 9 aa, Artificial Sequence
  Synthetic Construct
MVFFGNVDS

SEQ ID NO: 23 - protein, 6 aa, Artificial Sequence
  Synthetic Construct
QRRDRM

SEQ ID NO: 24 - protein, 6 aa, Artificial Sequence
  Synthetic Construct
QRRERM

SEQ ID NO: 25 - protein, 66 aa, Homo sapiens
SLYISQFIIMYSLDGKKWQTYRGNSTGTLMVFFGNVDSSGIKHNIFNPPIIARYIRLHPTHYSIRS

SEQ ID NO: 26 - protein, 45 aa, Homo sapiens
YSLDGKKWQTYRGNSTGTLMFFGNVDSSGIKNNIFNPPIIARYIR